(19) United States
(12) Patent Application Publication
(10) Pub. No.: US 2022/0186227 A1
(43) Pub. Date: Jun. 16, 2022

(54) Therapeutic Targets for Oncogenic KRAS-Dependent Cancers

(71) Applicant: University of Massachusetts, Boston, MA (US)

(72) Inventors: Michael R. Green, Boylston, MA (US); Dong-Hwan Kim, Shrewsbury, MA (US)

(21) Appl. No.: 17/439,439

(22) PCT Filed: Mar. 28, 2020
(86) PCT No.: PCT/US2020/024983

§ 371 Date: Sep. 15, 2021

Related U.S. Application Data
(60) Provisional application No. 62/823,964, filed on Mar. 26, 2019.

(51) Int. Cl. C12N 15/113; C07K 14/72; C12N 15/11

(52) U.S. Cl. CPC C12N 15/1135; C07K 14/721; C12N 15/1138; C12N 2310/20; C12N 2310/14; C12N 2310/531; C12N 15/111

(57) ABSTRACT

Compositions and methods for treating cancers containing an oncogenic KRAS mutant.

### CLAIM OF PRIORITY

This application claims the benefit of U.S. Provisional Patent Application Ser. No. 62/823,964, filed on Mar. 26, 2019. The entire contents of the foregoing are hereby incorporated by reference.

### TECHNICAL FIELD

Compositions and methods for treating cancers containing an oncogenic KRAS mutant are provided.

### BACKGROUND

RAS proteins are founding members of a large superfamily of small GTPases that serve as master regulators of signaling cascades involved in a wide range of cellular processes including proliferation, migration, adhesion, cytoskeletal integrity, survival and differentiation (Raj alingam et al. 2007 Biochem Biophys Acta 1773:1177-95). A common feature of RAS proteins is that they function in signal transduction across membranes, in particular in signaling induced by growth factors. RAS proteins require membrane association for their biological activity, and are attached to the membrane by virtue of post-translational farnesylation at the C-terminus of the protein (Ahearn et al. 2012, Nat Rev Mol Cell Biol 13:39-51).

### SUMMARY

There is no effective treatment of the major solid tumors containing oncogenic KRAS including lung cancer, colorectal cancer and pancreatic cancer. This study systematically identified factors that regulate KRAS expression. The results of this study have identified a novel class of potential therapeutic targets for the treatment of these oncogenic KRAS containing cancers. In particular, anti-androgens, which are FDA-approved for the treatment of prostate cancer, can be used to reduce KRAS levels in, and inhibit proliferation of, KRAS-dependent human cancer cells. Thus small molecule inhibitors for reducing KRAS levels can be used for the treatment of oncogenic KRAS-dependent cancers. Anti-androgens, such as apalutamide, can be used to treat oncogenic KRAS-dependent cancers both alone and in combination with conventional chemotherapeutic agents and immunotherapeutics.

Thus, provided herein are methods for treating a subject who has a cancer containing an oncogenic KRAS mutant. The methods include administering to the subject a therapeutically effective amount of an inhibitor of a KRAS-EF listed in Table 1, optionally in combination with a therapeutically effective amount of one or more chemotherapeutic and/or immunotherapeutic agents. Also provided are inhibitors of a KRAS-EF listed in Table 1, for use in a method of treating a subject who has a cancer containing an oncogenic KRAS mutant.

In some embodiments, the inhibitor is a small molecule antagonist of Androgen Receptor (AR), e.g., a non-steroidal antagonist, e.g., diarylthiohydantoin derivatives (e.g., apalutamide (Erleada, ARN-509), proxalutamide, enzalutamide (Xtandi), and RD-162), flutamide, nilutamide, bicalutamide, and topilutamide; AZD3514; darolutamide (ODM-201, BAY-1841788); a diarylhydantoin, e.g., 4-(hydroxymethyl)diarylhydantoin; a steroidal androgen receptor antagonist, e.g., a 17α-Hydroxyprogesterone derivative (e.g., cyproterone acetate, megestrol acetate, chlormadinone acetate, osaterone acetate); 19-Norprogesterone derivative (e.g., nomegestrol acetate); 19-Nortestosterone derivative (e.g., dienogest, oxendolone); or 17α-Spirolactone derivative (e.g., spironolactone, drospirenone); a progestin that has direct androgen receptor antagonistic activity (e.g., medrogestone, promegestone and trimegestone); an N-Terminal domain antiandrogen (e.g., bisphenol A, EPI-001, ralaniten, JN compounds); EZN-4176, AZD-5312, apatorsen, galeterone, ODM-2014, TRC-253, or BMS-641988.

In some embodiments, the inhibitor is a small molecule inhibitor of a KRAS-EF, e.g., as listed in Table A.

In some embodiments, the inhibitor is an inhibitory nucleic acid targeting a KRAS-EF, e.g., as listed in Table B. In some embodiments, the inhibitory nucleic acid is an antisense oligonucleotide, siRNA, or shRNA. The method of claim 5, wherein the inhibitory nucleic acid targets AR. The method of claim 1, wherein the inhibitory nucleic acid targets inhibits binding of AR to the KRAS promoter and/or first intron. In some embodiments, the inhibitory nucleic acid is a triplex forming oligo (TFO) that binds to the KRAS promoter and/or first intron. In some embodiments, the inhibitory nucleic acid comprises a decoy sequence that binds to AR.

In some embodiments, the inhibitor is a targeted protein degrader comprising a first ligand that binds to a KRAS-EF and a second ligand that binds to a E3 ubiquitin ligase, with a linker therebetween. In some embodiments, the targeted protein degrader is a PROTAC, e.g., ARV-110, ARD-69, ARD-61, or ARCC-4.

In some embodiments, the methods include identifying the subject as having a cancer containing an oncogenic KRAS mutant. In some embodiments, identifying the subject comprises determining the presence of a mutation in KRAS in the cancer. In some embodiments, determining the presence of a mutation comprises: obtaining a sample comprising a cell from the cancer; and detecting the presence of a mutation associated with cancer in a KRAS gene in the cell. In some embodiments, the mutation is G12, G13, and/or Q61.

Further, provided herein are methods for identifying a candidate compound for the treatment of a cancer containing an oncogenic KRAS mutant, comprising: providing a sample comprising a nucleic acid comprising a sequence comprising a promotor plus intron 1 of the KRAS gene, preferably promotor plus intron 1 of an oncogenic KRAS gene, and AR protein; contacting the sample with a test compound; measuring binding of the AR protein to the nucleic acid in the presence and absence of the test compound; and selecting a test compound that decreases binding of the AR to the nucleic acid as a candidate compound for the treatment of cancer containing an oncogenic KRAS mutant.

In some embodiments, the sample is a cell expressing a reporter construct comprising a fusion of a promotor plus intron 1 of an oncogenic KRAS gene and a detectable protein, e.g., a fluorescent protein.

Unless otherwise defined, all technical and scientific terms used herein have the same meaning as commonly understood by one of ordinary skill in the art to which this linvention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Construction of the endogenous KRAS-tdTomato reporter gene, and confirmation that shRNA-mediated knockdown of KRAS reduces expression of KRAS-tdTomato. (A) Construction of the endogenous KRAS-tdTomato reporter gene. CRISPR/Cas9-mediated homology directed repair was used to insert a tdTomato reporter at the 3′ end of exon 5 of KRAS, effectively deleting the tetrapeptide farnesylation signal sequence CVIM, in A549 cells. Insertion of the tdTomato reporter was selected using neomycin resistance, and the correct insertion site was confirmed by RT-PCR (data not shown). (B) qRT-PCR analysis monitoring expression of the KRAS-tdTomato reporter (using a forward primer in exon 4 of the KRAS gene and a reverse primer in tdTomato) in A549 cells harboring the reporter and expressing a non-silencing (NS) or KRAS shRNA. The results were normalized to that obtained with a NS shRNA, which was set to 1. Error bars indicate SD. (C) FACS analysis showing tdTomato fluorescence in parental A549 cells, and in A549 cells harboring the KRAS-tdTomato reporter and expressing a NS or KRAS shRNA.

FIG. 2. Schematic of the genome-wide CRISPR/Cas9-based screening strategy. Also shown are FACS plots of A549/KRAS-tdTomato cells before transduction with the CRISPR library, after transduction with the library, and after isolation and expansion of cells with tdTomato^{low} eGFP^{high} expression.

FIGS. 3A-C. Identification of factors that promote KRAS expression (KRAS-EFs) in A549 cells. (A) qRT-PCR analysis in A549 cells showing knockdown efficiencies of two independent shRNAs targeting KRAS-EFs identified from the primary CRISPR/Cas9-based screen. The results were normalized to that obtained with a NS shRNA, which was set to 1. Error bars indicate SD. (B) Immunoblot analysis showing KRAS levels in A549 cells expressing one of two independent shRNAs targeting a KRAS-EF, or as controls a NS or KRAS shRNA. α-tubulin (TUBA) was monitored as a loading control. (C) qRT-PCR analysis monitoring KRAS expression in A549 cells expressing one of two independent shRNAs targeting a KRAS-EF, or as controls a NS or KRAS shRNA. Error bars indicate SD.

FIG. 4. ShRNA-mediated knockdown of a KRAS-EF reduces proliferation of KRAS-dependent H358 human lung cancer cells. A549 and H358 cells were infected with lentiviruses expressing a NS, KRAS or KRAS-EF shRNA, and 24 hours after infection cells were treated with puromycin for three days. Cells were replenished with complete media without puromycin and cultured for two more days, and then stained with crystal violet.

FIGS. 5A-E. Small molecule inhibitors targeting AR, CLK2, PKCy, SENP7 and SOS1, reduce KRAS protein levels in A549 cells, and AR antagonists reduce proliferation of KRAS-dependent H358 human lung cancer cells. (A) Immunoblot analysis monitoring KRAS levels in A549 cells treated with the AR antagonist bicalutamide (0, 20, 80 μM for 3 days), the CLK2 inhibitor TG003 (50, 100, 200 μM for 2 days), the PKCy inhibitor Go 6983 (20, 40 80 μM for 2 days), the SENP7 inhibitor NSC 45551 (50, 100, 200 μM for 4 days) or the SOS1 inhibitor NSC 658497 (10, 20 μM for 4 days). (B, C) Immunoblot analysis monitoring KRAS protein levels (B) and qRT-PCR analysis monitoring KRAS expression (C) in A549 cells treated with the AR antagonist apalutamide (10, 20, 40, 80 μM for 3 days). Error bars indicate SD. (D, E) Cell proliferation assays in A549 and H358 cells treated with bicalutamide (20, 40, 60, 80 μM for 4 days) (D) or apalutamide (20, 40, 60 μM for 4 days).

FIGS. 6A-D. AR binds directly to the first intron of the KRAS gene. (A) Schematic of the KRAS promoter and first intron showing candidate AR-binding sites—either half-site androgen response elements (AGAACA) or 6-basepair AR-binding motifs (CCTTCT)—identified by bioinformatic analysis. (B) CMP analysis monitoring binding of the AR, or as a control IgG, to various regions in the KRAS promoter and first intron. Binding of AR in the presence of apalutamide was also monitored. (C) Schematic representations of the wild-type intron sequences and the deletion mutants generated. The boxed sequence represents the candidate AR-binding sites. (D) qRT-PCR monitoring KRAS expression (left) and immunoblot showing KRAS levels (right) upon deletion of regions in introns 1-1, 1-2 and 1-3. Error bars indicate SD.

FIGS. 7A-C. AR promotes KRAS expression through a ligand-independent signaling pathway. (A) Immunoblot analysis monitoring KRAS protein levels in A549 cells cultured in 10% FBS, 10% CS-FBS or under serum-free conditions (in which cells were grown in CS-FBS and serum starved for 24 hours). (B) Immunoblot analysis monitoring KRAS protein levels in A549 cells cultured in 10% CS-FBS and expressing a NS, KRAS or AR shRNA. (C) Immunoblot analysis monitoring KRAS protein levels in A549 cells cultured in 10% CS-FBS and treated with 10, 20 or 40 μM apalutamide for 3 days.

FIGS. 8A-J. Activation of AR is promoted by oncogenic KRAS and requires AKT1-mediated phosphorylation of AR. (A) Relative luciferase activity in isogenic H1975 KRAS(+/+) and H1975 KRAS(G12D/+) cell lines transfected with an AR reporter gene, in which the promoter of an AR target gene (ARR2, PSA or PSA 1210) was placed upstream of a luciferase reporter. The results were normalized to that obtained in H1975 KRAS(+/+) cells, which was set to 1. (B) qRT-PCR analysis of an endogenous AR target gene (FN1, HK2 or PSA) in H1975 KRAS(+/+) and H1975 KRAS(G12D/+) cells. (C)

Relative luciferase activity A549 cells expressing an NS or AKT1 shRNA and transfected with the AR reporter gene ARR2. (D, E) Immunoblot analysis showing KRAS levels in A549 cells expressing an NS or AKT1 shRNA (D) or treated with the AKT1 inhibitor MK2206 (E). (F) Immunoblot analysis. AR was immunoprecipitated from A549 cell extracts and immunoblotted using a phospho-S213 antibody or an AR antibody. (G) Immunoblot (left) and qRT-PCR (right) showing KRAS levels in wild-type A549 cells or A549 AR knockout (KO) cells. (H) Immunoblot showing KRAS levels in A549 AR KO cells expressing empty vector, wild-type AR, or the AR(S213A) mutant. (I) Immunoblot showing KRAS levels in H1975 KRAS(+/+) and H1975 KRAS(G12D/+) cells. (J) Model.

FIGS. 9A-C. The AR antagonist apalutamide kills oncogenic KRAS-dependent human lung cancer cell lines. (A) Relative cell viability of H1975 KRAS(+/+), H1975 KRAS(G12D/+) and H358 KRAS(G12C/+) cells treated with increasing concentrations of apalutamide for 3 days (left) or 8 days (right). (B) (Left) Immunoblot confirming increased levels of KRAS(G12V)-HA in H358 KRAS(G12C/+) cells stably expressing KAS(G12V) or empty vector. (Right) Relative cell viability of H358 KRAS(G12C/+) cells expressing vector or KRAS(G12V) and treated in the presence or absence of apalutamide (40 μM). (C) Relative cell viability of H358 KRAS(G12C/+) cells expressing vector or KRAS(G12V) and expressing an NS or AR shRNA. Error bars indicate SD.

FIGS. 10A-E. The AR antagonist apalutamide suppresses growth of oncogenic KRAS-dependent tumors. (A, B) Xenograft tumor formation assay. H358 KRAS(G12C/+) (C) and H1975 KRAS(+/+) (D) cells were subcutaneously injected into the flanks of nude mice (n=3), and when tumors reached 100 mm³, mice were treated with apalutamide (30 mg/kg) or vehicle once a day for the duration of the experiment, and tumor formation was measured every two days. (C-E) PDX tumor formation assay. Human lung PDXs containing KRAS(G12D/+) (C), KRAS(G12C/+) (D) or KRAS(+/+) (E) were transplanted into NSG mice (n=5) and when tumors reached 100 mm³, mice were treated daily with apalutamide (40 mg/kg/d) or vehicle, and tumor formation was measured. Shown are images of lung PDXs removed from mice on the last day of treatment. Error bars indicate SD.

FIGS. 11A-C. Confirmation of key results in KRAS-positive pancreatic cancer cells. (A) Immunoblot analysis showing KRAS levels in PANC-1 cells expressing one of two independent shRNAs targeting a subset of KRAS-EFs, or as controls a NS or KRAS shRNA. (B) Cell proliferation of HPAF-II cells treated with apalutamide (20, 40, 60 μM). (C) Xenograft tumor formation assay. HPAF-II cells were subcutaneously injected into the flanks of nude mice (n=3), and when tumors reached 100 mm³, mice were treated with apalutamide (30 mg/kg) or vehicle once a day for the duration of the experiment, and tumor formation was measured every two days. Error bars indicate SD.

FIGS. 12A-D. Confirmation of key results in KRAS-positive colorectal cancer cells. (A) Immunoblot analysis showing KRAS levels in HCT116 KRAS(G13D/+) cells expressing one of two independent shRNAs targeting a subset of KRAS-EFs, or as controls a NS or KRAS shRNA. (B) Relative cell viability of A549, HCT116 KRAS(G13D/+) or SW620 KRAS(G12V/G12V) cells treated with increasing concentrations of apalutamide for 3 (left) or 8 (right) days. (C) Xenograft tumor formation assay. SW620 KRAS(G12V/G12V) cells were subcutaneously injected into the flanks of nude mice (n=3), and when tumors reached 100 mm³, mice were treated with apalutamide (30 mg/kg) or vehicle once a day for the duration of the experiment, and tumor formation was measured every two days. (D) A human colorectal PDX containing KRAS(G13D/+) was transplanted into NSG mice (n=6) and when tumors reached 100 mm³, mice were treated daily with apalutamide (40 mg/kg/d) or vehicle, and tumor formation was measured. Shown are images of lung PDXs removed from mice on the last day of treatment. Error bars indicate SD.

### DETAILED DESCRIPTION

In the mammalian genome, there are three RAS genes: HRAS, KRAS and NRAS. These three RAS genes are the most commonly mutated oncogenes in human cancers, with KRAS being the most frequently mutated, accounting for approximately 85% of all RAS mutations in human tumors (Prior et al. 2012, Cancer Res 72:2457-67). Mutations in KRAS occur in approximately 98% of pancreatic ductal adenocarcinomas, 45% of colorectal carcinomas, 31% of lung adenocarcinomas and 23% of multiple myelomas (Cox et al. 2014, Nat Rev Drug Discov 13:828-51). Oncogenic mutants in KRAS occur predominantly at one of three residues (G12, G13 or Q61) and result in the impairment of intrinsic GTP hydrolysis activity, leading to constitutive activation of the protein.

Considerable experimental evidence indicates that in many cases continued expression of oncogenic KRAS is necessary for cellular proliferation and tumor growth. For example, RNA interference (RNAi)-mediated knockdown of KRAS impairs proliferation of human cancer cell lines containing oncogenic KRAS (henceforth referred to as KRAS-dependent cell lines) (Brummelkamp et al. 2002, Cancer Cell 2:243-7; Lim and Counter 2005, Cancer Cell 8:381-392; Singh et al 2009, Cancer Cell 15:489-500). Similarly, in mouse tumor models loss of oncogenic KRAS results in tumor regression and reduced metastasis (Chin et al. 1999, Nature 400:468-472; Collins et al 2012, PLoS ONE 7:e49707; Fisher et al. 2001, Genes Dev 15:3249-62; Ying et al. 2012, Cell 149:656-70; Boutin et al. 2017, Genes Dev 31:370-82; Yuan et al. 2018, Cell Reports 22:1889-1902). This phenomenon, known as “oncogene addiction”, suggests that oncogenic KRAS can not only initiate tumorigenesis but is also required for tumor maintenance. Notably, however, following genetic antagonism of KRAS function resistance can develop resulting in KRAS-independent cellular proliferation and tumor growth, which is likely due to the acquisition of alternative proliferative pathways (Kapoor et al. 2014, Cell 158:185-197; Muzumdar et al. 2017, Nat Commun 8:1090; Chen et al. 2018, Cancer Res 78:985-1002). Several studies have identified human and mouse cancer cell lines whose growth is not dependent on continued expression of KRAS (henceforth referred to as KRAS-independent cell lines) (Singh et al 2009, Cancer Cell 15:489-500; Yuan et al. 2018, Cell Reports 22:1889-1902; Muzumdar et al. 2017, Nat Commun 8:1090; Chen et al. 2018, Cancer Res 78:985-1002).

Because oncogenic KRAS can drive tumorigenesis, there has been great interest in developing antagonists of oncogenic KRAS function, including: (1) direct KRAS inhibitors, (2) inhibitors of KRAS membrane association, (3) inhibitors of downstream effector signaling pathways of KRAS, (4) inhibitors of KRAS synthetic lethal interaction partners, and (5) inhibitors of metabolic changes that occur in tumors containing oncogenic KRAS (Cox et al. 2014, Nat Rev Drug Discov 13:828-51). However, these approaches have been met with challenges. For example, the main barrier for developing direct small molecule inhibitors is that the KRAS GTP pocket is small and inaccessible due to high affinity for GTP. Recently, however, several groups have identified small molecules that selectively recognize and covalently bind the cysteine residue in the KRAS(G12C) mutant, locking it in an inactive state (Ostrem et al. 2013, Nature 503:548-51; Lito et al. 2016, Science 351:604-8; Patricelli et al. 2016, Cancer Discov 6:316-29; Janes et al. 2018, Cell 172:578-89). These inhibitors potently block KRAS(G12C)-dependent signal transduction and KRAS (G12C)-positive cancer cell viability in vitro and in mice. However, G12C is a relatively minor KRAS variant (accounting for ˜12% of oncogenic KRAS mutants), and it remains unclear whether similar covalent inhibitors can be identified for other, more common oncogenic KRAS mutants (Hobbs et al. 2016, Cancer Cell 29:251-3). In addition, inhibition of KRAS membrane association can be bypassed by alternative lipid modification pathways (such as geranylgeranylation) (Baker and Der 2013, Nature 497:577-578). Furthermore, inhibitors of KRAS effectors that affect downstream signaling pathways (e.g., RAF, MEK, ERK1/2) lack specificity and are prone to the development of resistance due to activation of parallel signaling pathways that promote cellular proliferation. To date, no KRAS inhibitor has been approved by the FDA.

We have taken a novel approach to identifying inhibitors of KRAS function. First, we have carried out a functional genomics screen to identify cellular factors that promote KRAS expression. Based upon this information, we then identified biological or small molecule inhibitors of the factors and pathways that promote KRAS expression, which substantially reduce KRAS expression in and proliferation of oncogenic KRAS-dependent human cancer cell lines expressing one of several different oncogenic KRAS mutants. The results of our study have identified a novel class of potential therapeutic targets for the treatment of oncogenic KRAS-dependent cancers. In particular, we show that FDA-approved androgen receptor antagonists, which are currently used to treat prostate cancer, reduce KRAS levels in oncogenic KRAS-dependent human cancer cell lines resulting in cell death, and suppress growth of tumors in mice derived from KRAS-positive human cancer cell lines or patient-derived xenografts.

Methods of Treatment

The methods described herein include methods for the treatment of cancers associated with mutations of KRAS, e.g., mutations that impair GAP-assisted GTP→GDP hydrolysis by KRAS. In some embodiments, the cancer is a solid tumor with a mutation in KRAS at G12, G13, and/or Q61. Mutations in KRAS occur in 97.7% of pancreatic ductal adenocarcinomas, 44.7% of colorectal adenocarcinomas, 30.9% of lung adenocarcinomas, 22.8% of multiple myelomas, and 21.4% of uterine corpus endometrioid carcinoma (Cox et al. 2014, Nat Rev Drug Discov 13:828-51). In addition, mutations in KRAS account for less than 20% of cases of skin cutaneous melanoma, uterine carcinosarcoma, thyroid carcinoma, stomach adenocarcinoma, acute myeloid leukemia, bladder urothelial carcinoma, cervical adenocarcinoma, head and neck squamous cell carcinoma, gastric carcinoma, esophageal adenocarcinoma, chronic lympocytic leukemia, lung squamous cell carcinoma, small cell lung carcinoma, renal papillary cell carcinoma, medulloblastoma and pilocytic astrocytoma, breast invasive carcinoma, hepatocellular carcinoma, cervical squamous cell carcinoma, ovarian serous adenocarcinoma, adrenocortical carcinoma, brain lower grade glioma, prostate adenocarcinoma, glioblastoma multiforme, and kidney renal clear cell carcinoma (Cox et al. 2014, Nat Rev Drug Discov 13:828-51). In some embodiments, the cancer is lung cancer, pancreatic cancer, or colorectal cancer. In some embodiments, the cancer is not prostate cancer, e.g., the subject has not been diagnosed with prostate cancer. Generally, the methods include administering a therapeutically effective amount of one or more inhibitors of a KRAS-EF as described herein, to a subject who is in need of, or who has been determined to be in need of, such treatment.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

As used in this context, to “treat” means to ameliorate at least one symptom of the cancer. Administration of a therapeutically effective amount of a compound described herein for the treatment of a cancer associated with oncogenic mutations in KRAS can result in one or more of decreased tumor size or growth rate or decreased tumor burden, and/or an increased life span or increased time to progression or reoccurrence.

In some embodiments, the methods can include a step of identifying a subject as having a cancer associated with mutations of KRAS, e.g., by obtaining a sample from the subject and detecting the presence of one or more mutations that impair GAP-assisted GTP→GDP hydrolysis by KRAS. In some embodiments, the mutation in KRAS is at G12, G13, and/or Q61 (Cox et al. 2014, Nat Rev Drug Discov 13:828-51; Prior et al. 2012, Cancer Res 72:2457-2467). As used herein the term “sample”, when referring to the material to be tested for the presence of a mutation, can include inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used may vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence of a mutation in KRAS. In some embodiments a technique suitable for the detection of alterations in the structure or sequence of nucleic acids, such as the presence of deletions, amplifications, or substitutions, can be used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

The methods can include using next generation sequencing or other methods to identify cancers with mutations in KRAS.

In some embodiments the methods include identifying and selecting a subject on the basis that they have a cancer with a mutation in KRAS.

Combination Therapies

The methods described herein can also include administering the KRAS-EF inhibitor in combination with other treatment modalities, e.g., chemotherapy or immunotherapy. For example, chemotherapy can include one or more agents used in XELOX or FOLFOX/FOLFIRI/FOLFOXRI or related regimens, e.g., fluorouracil (5-FU) (e.g., oral form capecitabine), preferably in combination with one or more of leucovorin, irinotecan and oxaliplatin; luoropyrimidine such as 5-FU or capecitabine; irinotecan or oxaliplatin in combination with a fluoropyrimidine; or EMICORON (Porru et al., J Exp Clin Cancer Res. 2018; 37: 57). Immunotherapies can include checkpoint inhibitors, e.g., as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., Histol Histopathol. 2007 June; 22(6):687-96; Eggermont et al., Semin Oncol. 2010 October; 37(5):455-9; Klinke D J., Mol Cancer. 2010 Sep. 15; 9:242; Alexandrescu et al., J Immunother. 2010 July-August; 33(6):570-90; Moschella et al., Ann N Y Acad Sci. 2010 April; 1194:169-78; Ganesan and Bakhshi, Natl Med J India. 2010 January-February; 23(1):21-7; Golovina and Vonderheide, Cancer J. 2010 July-August; 16(4):342-7. Preferably, agents that target EGFR are not used.

In some embodiments, e.g., wherein the subject has colorectal cancer, 5-Fluorouracil (5-FU) (e.g., oral form capecitabine), preferably in combination with one or more of leucovorin, irinotecan and oxaliplatin, e.g., in a FOLFOX/FOLFIRI/FOLFOXRI regimen; panitumumab, cetuximab, bevacizumab, ramucirumab, and aflibercept can also be combined with 5-FU, plus irinotecan or oxaliplatin, for metastatic colorectal cancer. Regorafenib can also be used in the present methods.

In some embodiments, e.g., wherein the subject has pancreatic cancer, gemcitabine, 5-Fluorouracil (5-FU) (e.g., oral form capecitabine), preferably in combination with one or more of leucovorin, irinotecan and oxaliplatin, e.g., in a FOLFOX/FOLFIRI/FOLFOXRI regimen; paclitaxel (e.g., ABRAXANE® (albumin-bound)), and/or irinotecan (ONIVYDE®, liposome injection) can be used.

In some embodiments, e.g., wherein the subject has non-small cell lung cancer, the methods can include administering chemotherapy comprising one, two, or more of Cisplatin; Carboplatin; Paclitaxel (Taxol); Albumin-bound paclitaxel (nab-paclitaxel, Abraxane); Docetaxel (Taxotere); Gemcitabine (Gemzar); Vinorelbine (Navelbine); Irinotecan (Camptosar); Etoposide (VP-16); Vinblastine; and Pemetrexed (Alimta). Some preferred combinations include cisplatin or carboplatin plus one other drug, or gemcitabine with vinorelbine or paclitaxel. Targeted therapy drugs including bevacizumab (Avastin), ramucirumab (Cyramza), or necitumumab (Portrazza) can be added as well.

Inhibitors of KRAS-EF

The methods can include administering one or more inhibitors of one or more KRAS-EFs as described herein. The inhibitors can be, e.g., small molecules, protein degraders, or inhibitory nucleic acids.

TABLE A
Small Molecule Inhibitors of KRAS protein
expression promoting factors (KRAS-EFs)

| | | Source |
|---|---|---|
| Gene | | (commercial, |
| symbol | Available KRAS-EF small molecule inhibitors | academic, other) |
| AR | See below | Commercial |
| ATF7IP | — | |
| BRI3BP | — | |
| CASP8 | Z-IETD-FMK | Commercial |
| CLK2 | TG003 (also inhibits other CLK family members | Commercial |
| | CLK1 and CLK4) | |
| DEPDC7 | — | |
| EBF1 | — | |
| GEMIN4 | — | |
| KHDC4 | — | |
| (aka | | |
| BLOM7) | | |
| IFI16 | — | |
| MMP8 | CAS 236403-25-1/MMP-8 inhibitor I | Commercial |
| NKIRAS1 | — | |
| PAGE1 | — | |
| PARP14 | Compounds “8k” and “8m” | Academic |
| | | (Holechek et al. |
| | | 2018, Bioorg Med |
| | | Chem Lett |
| | | 28: 2050-2054) |
| POLR2K | α-amanatin (broad RNA pol II inhibitor) | Commercial |
| PRKCG | Gö 6983, Phorbol 12-myristate 13-acetate, K-252c, | |
| | Calphostin (also inhibit other PKC family members) | |
| PYM1 | — | |
| (aka | | |
| WIBG) | | |
| RACK1 | — | |
| (aka | | |
| GNB2L1) | | |
| RRAGD | — | |
| RRP12 | — | |
| SENP7 | NSC 45551 | NCI Developmental |
| | | Therapeutics |
| | | Program |
| SOS1 | NSC 658497 | NCI Developmental |
| | | Therapeutics |
| | | Program |
| STYK1 | — | |
| TICAM1 | — | |
| TNIK | NCB-0846, N5355, KY-05009 | Commercial and |
| | | academic (Uno et |
| | | al. 2016, EJ Cancer |
| | | 69(supp1): S38 |
| | | (abstract) |
| TGFB2 | SB 431542, A83-01 | Academic (Halder |
| | (inhibitors of TGF-β receptor kinases) or | et al. 2005, |
| | Pirfenidone (inhibits TGF-β production) | Neoplasia 7: 509-521) |
| | | and commercial |
| YES1 | PD173955, PRT062607, AZD0530 (also inhibit | Commercial |
| | other kinases such as Src, Abl and Syk) | |
| ZNF74 | — | |

A number of AR antagonists (sometimes also referred to as antiandrogens) that directly target the AR rather than the ligand androgen are known in the art. These include nonsteroidal androgen receptor antagonists, e.g., diarylthiohydantoin derivatives apalutamide (Erleada, ARN-509), proxalutamide, enzalutamide (Xtandi), and RD-162, as well as the related flutamide, nilutamide, bicalutamide, and topilutamide; AZD3514 (Omlin et al., Invest New Drugs. 2015 June; 33(3):679-90); darolutamide (ODM-201, BAY-1841788) (Shore, Expert Opin Pharmacother. 2017 June; 18(9):945-952); and diarylhydantoins, e.g., 4-(hydroxymethyl)diarylhydantoin (see, e.g., Nique et al., J Med Chem. 2012 Oct. 11; 55(19):8225-35; Nique et al., J Med Chem. 2012 Oct. 11; 55(19):8236-47; EP2444085B1). Steroidal androgen receptor antagonists include 17α-Hydroxyprogesterone derivatives (e.g., cyproterone acetate, megestrol acetate, chlormadinone acetate, osaterone acetate); 19-Norprogesterone derivatives (e.g., nomegestrol acetate); 19-Nortestosterone derivatives (e.g., dienogest, oxendolone); 17α-Spirolactone derivatives (e.g., spironolactone, drospirenone); some progestins (e.g., some listed above as well as medrogestone, promegestone and trimegestone) that have direct androgen receptor antagonistic activity; and N-Terminal domain antiandrogens (e.g., bisphenol A, EPI-001, ralaniten, JN compounds). Others can include EZN-4176, AZD-5312, apatorsen, galeterone, ODM-2014, TRC-253, and BMS-641988.

See, e.g., WO2011106570A1; U.S. Pat. Nos. 9,439,912; 10,155,006; 9,216,957; Elshin et al., Med Res Rev. 2018 Nov. 22. doi: 10.1002/med.21548; Rathkopf and Scher, Cancer J. 2013 January-February; 19(1): 43-49; Ferroni and Varchi, Curr Med Chem. 2018 Sep. 12. doi: 10.2174/0929867325666180913095239; Dellis and Papatsoris, Expert Opin Pharmacother. 2019 February; 20(2):163-172. and Mohler et al., “Androgen receptor antagonists: a patent review (2008-2011).” Expert opinion on therapeutic patents 22 5 (2012): 541-65.

Targeted Protein Degraders

In some embodiments, the inhibitor is a targeted protein degrader. Protein degraders are small molecules that have two active ends; one binds to a KRAS-EF, and one that binds to a protease, e.g., E3 ubiquitin ligase (see, e.g., Jarvis, C&EN, 96(8) 2018; Watt et al., “Targeted protein degradation in vivo with Proteolysis Targeting Chimeras: Current status and future considerations,” Drug Discovery Today: Technologies, 2019, doi.org/10.1016/j.ddtec.2019.02.005; Zhang et al., “Targeted protein degradation mechanisms,” Drug Discovery Today: Technologies, 2019, doi.org/10.1016/j.ddtec.2019.01.001; Pettersson and Crews, “PROteolysis TArgeting Chimeras (PROTACs)—Past, present and future,” Drug Discovery Today: Technologies, 2019, doi.org/10.1016/j.ddtec.2019.01.002). For example, ARV-110 is an AR protein degrader developed by Arvinas (arvinas.com/therapeutic-programs/androgen-receptor). Other AR protein degraders include ARD-69 (Han et al. 2019, J Med Chem 62:941-964); ARD-61 (Kregel et al., Neoplasia. 2020 Jan. 10; 22(2):111-119); and ARCC-4 (Salami et al. 2018, Commun Biol 1:100); a non-peptidic PROTAC (proteolysis-targeting chimera) for AR is described in Schneekloth et al. 2008, Bioord Med Chem Lett 18:5904-5908.

Inhibitory Nucleic Acids

Inhibitory nucleic acids useful in the present methods and compositions include antisense oligonucleotides, ribozymes, siRNA compounds, single-or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target KRAS-EF nucleic acid and modulate its function; see Table B for exemplary sequences of KRAS-EFs. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); or a short, hairpin RNA (shRNA); or combinations thereof. See, e.g., WO 2010040112.

TABLE B
Exemplary human KRAS-EF sequences

| Gene | | GenBank RefSEQ ID. |
|---|---|---|
| symbol | Gene Name/isoform | (transcript → protein) |
| AR | androgen receptor/isoform 1 | NM_000044.4 → NP_000035.2 |
| | androgen receptor/isoform 2 | NM_001011645.3 → NP_001011645.1 |
| | androgen receptor/isoform 3 | NM_001348061.1 → NP_001334990.1 |
| | androgen receptor/isoform 4 | NM_001348063.1 → NP_001334992.1 |
| | androgen receptor/isoform 5 | NM_001348064.1 → NP_001334993.1 |
| ATF7IP | activating transcription factor 7- | NM_181352.1 → NP_851997.1 |
| | interacting protein 1/isoform 1 | |
| | activating transcription factor 7- | NM_018179.4 → NP_060649.3 |
| | interacting protein 1/isoform 2 | |
| | activating transcription factor 7- | NM_001286514.1 → NP_001273443.1 |
| | interacting protein 1/isoform 3 | |
| | activating transcription factor 7- | NM_001286515.1 → NP_001273444.1 |
| | interacting protein 1/isoform 4 | |
| BRI3BP | BRI3-binding protein precursor | NM_080626.6 → NP_542193.3 |
| CASP8 | caspase-8/isoform A precursor | NM_001228.4 → NP_001219.2 |
| | caspase-8/isoform B precursor | NM_033355.3 → NP_203519.1 |
| | caspase-8/isoform C precursor | NM_033356.3 → NP_203520.1 |
| | caspase-8/isoform E precursor | NM_033358.3 → NP_203522.1 |
| | caspase-8/isoform G precursor | NM_001080125.1 → NP_001073594.1 |
| CLK2 | dual specificity protein kinase CLK2/ | NM_001294338.2 → NP_001281267.1 |
| | isoform 1 | |
| | dual specificity protein kinase CLK2/ | NM_003993.3 → NP_003984.2 |
| | isoform 2 | |
| | dual specificity protein kinase CLK2/ | NM_001294339.1 → NP_001281268.1 |
| | isoform 3 | |
| | dual specificity protein kinase CLK2/ | NM_001363704.1 → NP_001350633.1 |
| | isoform 4 | |
| DEPDC7 | DEP domain-containing protein 7/ | NM_001077242.2 → NP_001070710.1 |
| | isoform 1 | |
| | DEP domain-containing protein 7/ | NM_139160.2 → NP_631899.2 |
| | isoform 2 | |
| EBF1 | transcription factor COE1/isoform 1 | NM_001290360.2 → NP_001277289.1 |
| | transcription factor COE1/isoform 2 | NM_024007.5 → NP_076870.1 |
| | transcription factor COE1/isoform 3 | NM_182708.2 → NP_874367.1 |
| | transcription factor COE1/isoform 4 | NM_001324101.1 → NP_001311030.1 |
| | transcription factor COE1/isoform 5 | NM_001324103.1 → NP_001311032.1 |
| | transcription factor COE1/isoform 6 | NM_001324106.1 → NP_001311035.1 |
| | transcription factor COE1/isoform 7 | NM_001324107.1 → NP_001311036.1 |
| | transcription factor COE1/isoform 8 | NM_001324108.1 → NP_001311037.1 |
| | transcription factor COE1/isoform 9 | NM_001324109.1 → NP_001311038.1 |
| | transcription factor COE1/isoform 10 | NM_001324111.1 → NP_001311040.1 |
| | transcription factor COE1/isoform 11 | NM_001364155.1 → NP_001351084.1 |
| | transcription factor COE1/isoform 12 | XM_024454395.1 → XP_024310163.1 |
| | transcription factor COE1/isoform 13 | NM_001364156.1 → NP_001351085.1 |
| | transcription factor COE1/isoform 14 | NM_001364157.1 → NP_001351086.1 |
| | transcription factor COE1/isoform 15 | NM_001364158.1 → NP_001351087.1 |
| | transcription factor COE1/isoform 16 | NM_001364159.1 → NP_001351088.1 |
| GEMIN4 | | |
| KHDC4 | KH homology domain-containing | NM_014949.4 → NP_055764.2 |
| (aka | protein 4 | |
| BLOM7) | | |
| IFI16 | gamma-interferon-inducible protein | NM_001206567.1 → NP_001193496.1 |
| | 16/isoform 1 | |
| | gamma-interferon-inducible protein | NM_005531.2 → NP_005522.2 |
| | 16/isoform 2 | |
| | gamma-interferon-inducible protein | NM_001364867.1 → NP_001351796.1 |
| | 16/isoform 3 | |
| MMP8 | neutrophil collagenase/isoform 1 | NM_002424.3 → NP_002415.1 |
| | preproprotein | |
| | neutrophil collagenase/isoform 2 | NM_001304441.1 → NP_001291370.1 |
| NKIRAS1 | NF-kappa-B inhibitor-interacting Ras- | NM_020345.3 → NP_065078.1 |
| | like protein 1 | |
| PAGE1 | P antigen family member 1 | NM_003785.4 → NP_003776.2 |
| PARP14 | protein mono-ADP-ribosyltransferase | NM_017554.3 → NP_060024.2 |
| | PARP14 | |
| POLR2K | DNA-directed RNA polymerases I, II, | NM_005034.4 → NP_005025.1 |
| | and III subunit RPABC4 | |
| PRKCG | protein kinase C gamma type/isoform 1 | NM_001316329.1 → NP_001303258.1 |
| | protein kinase C gamma type/isoform 2 | NM_002739.5 → NP_002730.1 |
| PYM1 | partner of Y14 and mago/isoform 1 | NM_032345.3 → NP_115721.1 |
| (aka | partner of Y14 and mago/isoform 2 | NM_001143853.1 → NP_001137325.1 |
| WIBG) | | |
| RACK1 | receptor of activated protein C kinase 1 | NM_006098.4 → NP_006089.1 |
| (aka | | |
| GNB2L1) | | |
| RRAGD | ras-related GTP-binding protein D | NM_021244.4 → NP_067067.1 |
| RRP12 | RRP12-like protein/isoform 1 | NM_015179.3 → NP_055994.2 |
| | RRP12-like protein/isoform 2 | NM_001145114.1 → NP_001138586.1 |
| | RRP12-like protein/isoform 3 | NM_001284337.1 → NP_001271266.1 |
| SENP7 | sentrin-specific protease 7/isoform 1 | NM_020654.5 → NP_065705.3 |
| | sentrin-specific protease 7/isoform 2 | NM_001077203.2 → NP_001070671.1 |
| SOS1 | son of sevenless homolog 1 | NM_005633.3 → NP_005624.2 |
| STYK1 | NM_018423.3 → NP_060893.2 | tyrosine-protein kinase STYK1 |
| TICAM1 | TIR domain-containing adapter | NM_182919.3 → NP_891549.1 |
| | molecule 1 | |
| TNIK | TRAF2 and NCK-interacting protein | NM_015028.4 → NP_055843.1 |
| | kinase/isoform 1 | |
| | TRAF2 and NCK-interacting protein | NM_001161560.2 → NP_001155032.1 |
| | kinase/isoform 2 | |
| TGFB2 | transforming growth factor beta-2 | NM_001135599.3 → NP_001129071.1 |
| | proprotein/isoform 1 precursor | |
| | transforming growth factor beta-2 | NM_003238.5 → NP_003229.1 |
| | proprotein/isoform 2 preproprotein | |
| YES1 | tyrosine-protein kinase Yes | NM_005433.4 → NP_005424.1 |
| ZNF74 | zinc finger protein 74/isoform a | NM_003426.4 → NP_003417.2 |
| | zinc finger protein 74/isoform b | NM_001256523.1 → NP_001243452.1 |

Note that for some of the above, additional isoforms exist that can be identified informatically, e.g., in the NCBI GenBank database.

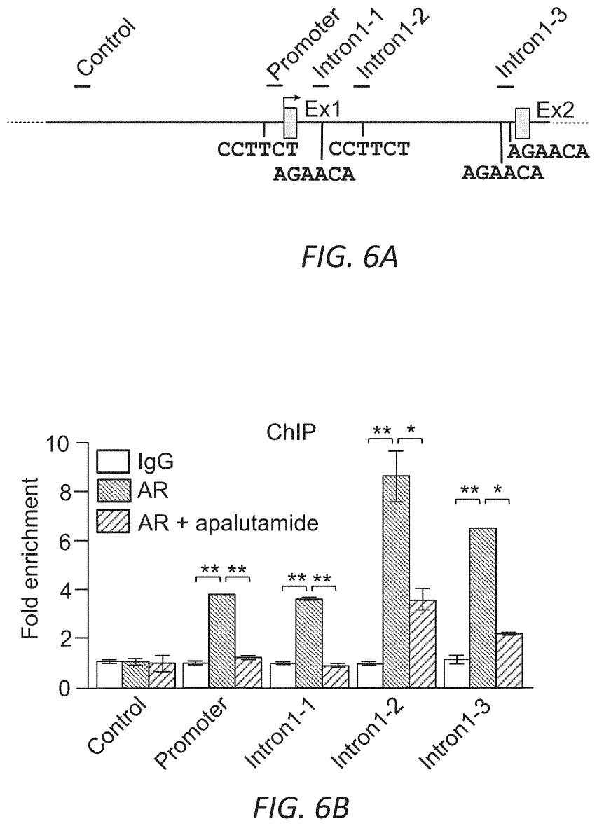

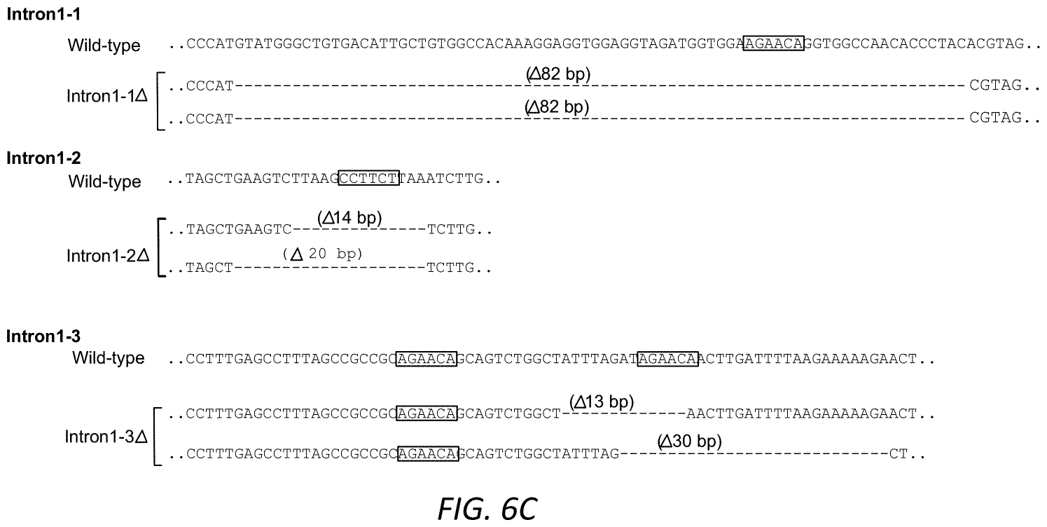

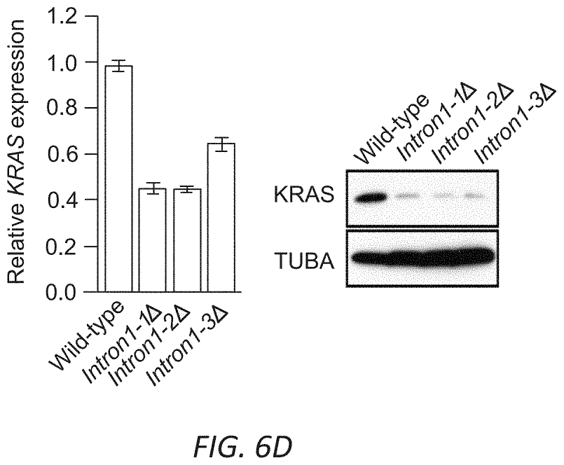

In some embodiments, the inhibitory nucleic acids are KRAS-binding oligos that disrupt binding of AR to the KRAS promotor (see, e.g., FIG. 6A), e.g., decoy oligos that comprise multiple optimized AR-binding site sequences, e.g., at least 80%, 90%, 95 identical to the 15-basepair SELEX-derived ARE, AGAACATCTCGTGTACC (SEQ ID NO:1); the 15-basepair in vivo-derived inverted repeat (IR)-ARE, AGAACAGCAAGTACT (SEQ ID NO:2); and the 15-basepair in vivo-derived direct repeat (DR)-ARE, AGAACTGGAAGAGCT (SEQ ID NO:3).

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Rib ozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261 :1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 Nov; 60(9):633-8; Ørom et al., Gene. 2006 May 10; 372( ):137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH,˜N(CH3)˜O˜CH2 (known as a methylene(methylimino) or MMI backbone], CH2-O—N (CH3)-CH2, CH2-N (CH3)-N (CH3)-CH2 and O—N (CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones;

and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH3O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2;

heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, Helv. Chim. Acta, 1995, 78, 486). Other preferred modifications include 2′-methoxy (2′-0-CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1 ,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammoniuml,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herien.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Triplex-Forming Oligonucleotides

In some embodiments, the oligonucleotides are triplex-forming oligonucleotides (TFOs) that bind to the KRAS promoter and/or intron 1. TFOs are defined as triplex-forming oligonucleotides which bind as third strands to duplex DNA in a sequence specific manner. Triplex-forming oligonucleotides may be comprised of any possible combination of nucleotides and modified nucleotides. Modified nucleotides may contain chemical modifications of the heterocyclic base, sugar moiety or phosphate moiety. TFOs, and methods of making them, are known in the art; see, e.g., Frank-Kamenetskii and Mirkin, Annual Review of Biochemistry, 64:65-95 (1995); Vasquez and Glazer, Quarterly Reviews of Biophysics, 35(01):89-107 (2002); US PGPub Nos. 20070219122; US20110130557; and US20090216003. In general, the TFO is a single-stranded nucleic acid molecule between 5 and 100 nucleotides in length, preferably between 7 and 40 nucleotides in length, e.g., 10 to 20 or 20 to 30 nucleotides in length. In some embodiments, the base composition is homopurine or homopyrimidine, polypurine or polypyrimidine. The oligonucleotides can be generated using known DNA synthesis procedures.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/ generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Patent No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Pharmaceutical Compositions

The methods described herein can include the administration of pharmaceutical compositions and formulations comprising inhibitory nucleic acid sequences designed to target an RNA.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g.,

U.S. Patent No. 5,716,928 describing using essential oils or essential oil components for increasing bioavailability and reducing inter-and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono-or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krutzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Methods of Screening

Included herein are methods for screening test compounds, e.g., polypeptides, polynucleotides, inorganic or organic large or small molecule test compounds, to identify agents useful in the treatment of cancers associated with KRAS mutations, e.g., as described herein.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell or cell-free sample, comprising a nucleic acid comprising a KRAS promoter and/or first intron sequence and purified AR, under conditions wherein the AR can bind to the nucleic acid. Binding in the presence and absence of the test compound is evaluated, e.g., using methods known in the art.

A test compound that has been screened by a method described herein and determined to disrupt or reduce AR binding to the KRAS promoter and/or first intron can be considered a candidate compound. A candidate compound that has been screened, e.g., in an in vivo model of a disorder, e.g., a xenograft model using human cancer cells containing an oncogenic KRAS mutant, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that disrupt or reduce AR binding to the KRAS promoter and/or first intron, and/or reduce tumor size, tumor growth, and/or tumor growth rate) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating cancers associated with KRAS mutations, e.g., as described herein. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a cancer associated with KRAS mutations, as described herein. The animal can be monitored for a change in the disorder, e.g., for an improvement in a parameter of the disorder, e.g., a parameter related to clinical outcome. In some embodiments, the parameter is tumor size or growth rate and an improvement would be decreased tumor size and/or growth rate.

Pharmaceutical Compositions and Methods of Administration

The methods described herein include the use of pharmaceutical compositions comprising at least one inhibitor of a KRAS-EF (e.g., a KRAS-EF as listed in Table 1) as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., chemotherapeutics or immunotherapeutics, e.g., as known in the art and/or described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1. Identification of Androgen Receptor, and Other Factors that Promote KRAS Expression, as Therapeutic Targets for Oncogenic KRAS-Dependent Cancers

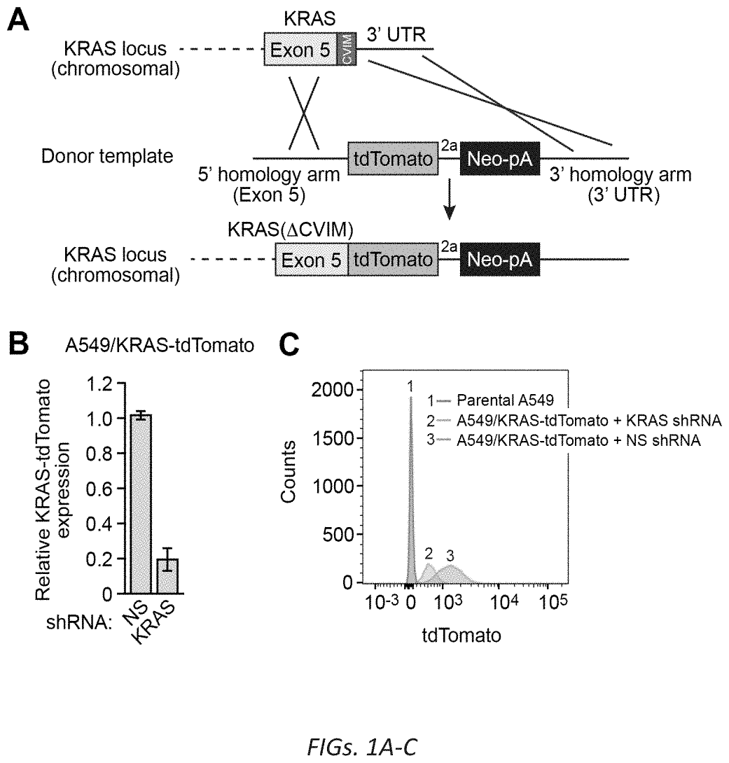

To identify cellular factors that promote KRAS expression, we performed a genome-scale CRISPR/Cas9-based screen to identify genes that, when knocked out, would reduce the levels of KRAS. To facilitate high-throughput screening, we used CRISPR/Cas9-mediated homology-directed repair to construct a reporter cell line in which the endogenous KRAS gene was fused at its 3′ end to the fluorescent reporter tdTomato (FIG. 1A). We constructed the fusion reporter to delete the 3′ terminal tetrapeptide farnesylation signal sequence CVIM because the farnesylation reaction involves a proteolytic cleavage (Manlaridis et al. 2013, Nature 504:301-5) that would separate KRAS from the tdTomato reporter. We constructed the reporter cell line in A549 human lung cancer cells, which harbor a homozygous KRAS(G12S) oncogenic mutation but are not dependent on KRAS for viability and proliferation (Singh et al. 2009, Cancer Cell 15:489-500) and therefore are not killed following reduction of KRAS(G12S) levels. The reporter cell line also expresses a control fluorophore, enhanced green fluorescence protein (eGFP), driven by a constitutive CMV promoter, allowing us to exclude factors whose knockout leads to a general reduction in protein levels or cell survival, which would also result in decreased eGFP signal. Cells harboring a gene knockout that selectively reduces expression of KRAS could be identified as tdTomato^{low} eGFP^{high}. We confirmed that small hairpin RNA (shRNA)-mediated knockdown of KRAS in these cells reduced expression of the KRAS-tdTomato reporter (FIG. 1B) and tdTomato fluorescence (FIG. 1C).

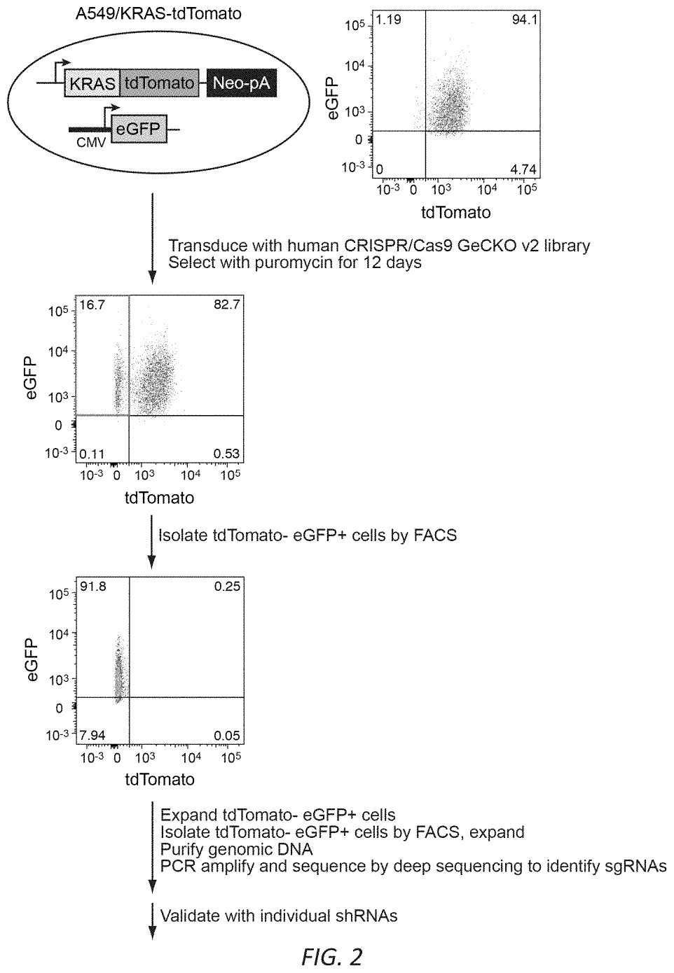

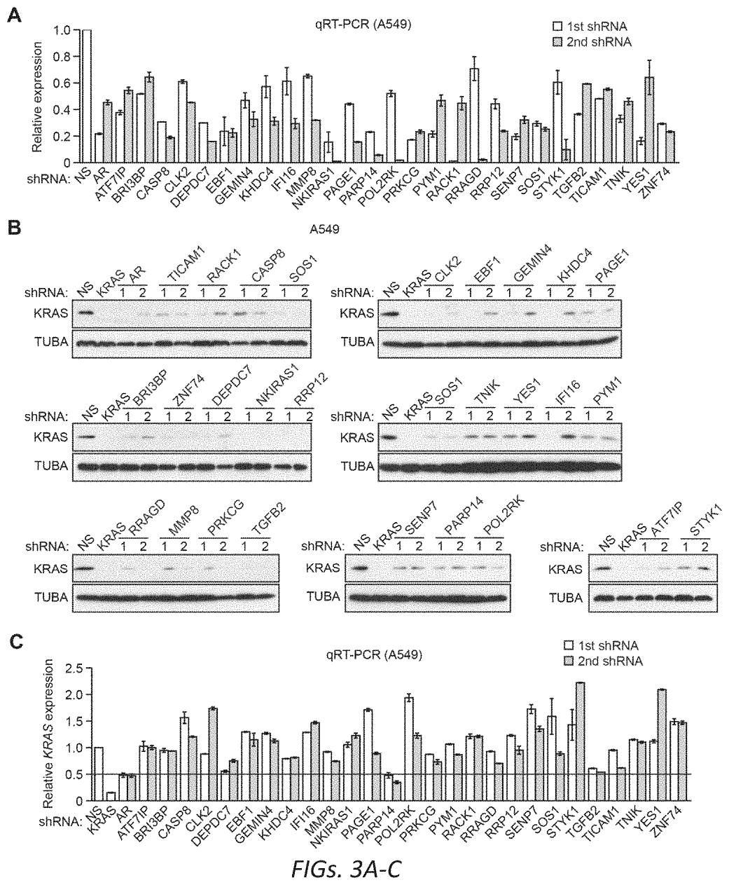

We transfected the reporter cell line with the human CRISPR/Cas9 GeCKO v2 library (Addgene), which consists of ˜123,000 single guide RNAs (sgRNAs) targeting ˜19,000 genes, with ˜six sgRNAs per gene (Sanjana et al. 2014, Nat Methods 11:783-4) (FIG. 2). Cells were puromycin selected for 12 days, tdTomato^{low} eGFP^{high} cells were isolated by fluorescent activated cell sorting (FACS) and expanded, and sgRNAs were identified by deep sequencing. Using this approach, we identified 35 genes that had five or more independent sgRNAs that were significantly enriched in the tdTomato^{low} eGFP^{high} population relative to sgRNAs present in the tdTomato^{low} eGFP^{high} population. To validate the candidate genes, we knocked down each gene in parental A549 cells using two independent shRNAs (FIG. 3A) and monitored endogenous KRAS protein levels by immunoblotting. We considered a candidate validated if both shRNAs (1) decreased KRAS protein levels and (2) decreased mRNA levels of the target gene compared to that obtained with a control non-silencing (NS) shRNA. This approach enabled us to identify 28 factors that promote KRAS expression (FIG. 3B and see Table 1). For convenience, we refer to the factors that promote KRAS expression as KRAS expression factors (KRAS-EFs). These 28 KRAS-EFs include transcription factors, pre-mRNA splicing regulators, mRNA stability factors, and regulators of signaling pathways. For several of these KRAS-EFs, there are small molecule inhibitors that are either commercially available or can be provided by an academic laboratory or other research institution (Table 1). The sequences of two independent shRNAs targeting each of the 28 KRAS-EFs are shown in Table 2.

TABLE 1
List of the 28 KRAS protein expression promoting factors (KRAS-EFs)

| Gene | | |
|---|---|---|
| symbol | Gene name | Function |
| AR | androgen receptor | Steroid hormone receptor that regulates |
| | | gene expression |
| ATF7IP | activating transcription | Recruiter that couples transcription |
| | factor 8 interacting protein | factors to the general transcription |
| | | apparatus. Can act as an activator or |
| | | repressor, depending on context. |
| BRI3BP | BRI3 binding protein | Involved in tumorigenesis and may |
| | | function by stabilizing p53/TP53 |
| CASP8 | caspase 8 | Most upstream protease of the activation |
| | | cascade of caspases; cleaves and activates |
| | | CASP3, CASP4, CASP6, CASP7, |
| | | CASP9, CASP10. |
| CLK2 | CDC like kinase 2 | Dual specificity kinase. Phosphorylates |
| | | SR proteins of the spliceosome complex. |
| DEPDC7 | DEP domain containing 7 | GTPase activator |
| EBF1 | EBF transcription factor 1 | Transcriptional activator |
| GEMIN4 | gem nuclear organelle | Part of the SMN complex, which plays a |
| | associated protein 4 | role in the assembly of snRNPs. |
| KHDC4 | KH domain containing 4, | RNA-binding protein involved in pre- |
| (aka | pre-mRNA splicing factor | mRNA splicing |
| BLOM7) | | |
| IFI16 | interferon gamma inducible | Putative transcriptional regulator; |
| | protein 16 | modulates the function of p53/TP53 |
| MMP8 | matrix metalloproteinase 8 | Cleaves interstitial collagens |
| NKIRAS1 | NFKB inhibitor interacting | Atypical Ras-like protein that acts as a |
| | Ras like 1 | potent regulator of NF-kappa-B activity |
| PAGE1 | PAGE family member 1 | P antigen family member that contains an |
| | | antigenic peptide that is recognized by |
| | | cytotoxic T cells |
| PARP14 | poly(ADP-ribose) | ADP-ribosyltransferase known to mono- |
| | polymerase family member | ADP-ribosylate STAT1, STAT6, PARP9 |
| | 14 | |
| POLR2K | RNA polymerase II subunit | Common component of RNA |
| | K | polymerases I, II and III. |
| PRKCG | protein kinase c gamma | Calcium-activated, phospholipid- and |
| | | diacylglycerol (DAG)-dependent |
| | | serine/threonine-protein kinase that plays |
| | | diverse roles in neuronal cells and eye |
| | | tissues |
| PYM1 | PYM homolog 1, exon | Key regulator of the exon junction |
| (aka | junction complex associated | complex (EJC), which plays a role in |
| WIBG) | factor | directing post-transcriptional processes in |
| | | the cytoplasm such as mRNA export, |
| | | nonsense-mediated mRNA decay (NMD) |
| | | or translation. |
| RACK1 | receptor for activated C | Scaffolding protein involved in the |
| (aka | kinase 1 | recruitment, assembly and/or regulation |
| GNB2L1) | | of a variety of signaling molecules. |
| RRAGD | Ras related GTP binding D | Guanine nucleotide-binding protein |
| | | forming heterodimeric Rag complexes |
| | | involved in activation of TOR signaling |
| RRP12 | ribosomal RNA processing | RNA-binding protein |
| | 12 homolog | |
| SENP7 | SUMO specific peptidase 7 | Protein that deconjugates SUMO2 and |
| | | SUMO3 from target proteins. |
| SOS1 | SOS Ras/Rac guanine | Promotes the exchange of Ras-bound |
| | nucleotide exchange factor 1 | GDP by GTP |
| STYK1 | serine/threonine/tyrosine | Probable tyrosine protein kinase with |
| | kinase 1 | strong transforming capabilities on a |
| | | variety of cell lines. |
| TICAM1 | toll-like receptor adapter | Adapter used by TLR3 and TLR4 to |
| | molecule 1 | mediate NFKB and IRF activation, and to |
| | | induce apoptosis. |
| TNIK | TRAF2 and NICK | Serine/threonine kinase that acts as an |
| | interacting kinase | essential activator of the Wnt signaling |
| | | pathway. |
| TGFB2 | transforming growth factor | Multifunctional protein that regulates |
| | beta 2 | various processes such as angiogenesis |
| | | and heart development. |
| YES1 | YES proto-oncogene 1, Src | Non-receptor protein tyrosine kinase that |
| | family tyrosine kinase | is involved in the regulation of cell |
| | | growth and survival, apoptosis, cell-cell |
| | | adhesion, cytoskeleton remodeling, and |
| | | differentiation. |
| ZNF74 | zinc finger protein 74 | May play a role in RNA metabolism |

TABLE 2
Sequences of two independent
shRNAs targeting each of the 28 KRAS-EFs

| Gene | | | SEQ |
|---|---|---|---|
| symbol | Oligo ID | Full Hairpin Sequence | ID NO: |
| AR | TRCN0000003715 | CCGGCCTGCTAATCAAGTCACACATCTCGA | 4. |
| | | GATGTGTGACTTGATTAGCAGGTTTTT | |
| | TRCN0000003717 | CCGGCGCGACTACTACAACTTTCCACTCGA | 5. |
| | | GTGGAAAGTTGTAGTAGTCGCGTTTTT | |
| ATF7IP | TRCN0000020828 | CCGGCGTCGATATATGGAAGAAGAACTCGA | 6. |
| | | GTTCTTCTTCCATATATCGACGTTTTT | |
| | TRCN0000020825 | CCGGCCAGGGACTTTGGTGACTAATCTCGA | 7. |
| | | GATTAGTCACCAAAGTCCCTGGTTTTT | |
| BRI3BP | V2LHS_71753 | TGCTGTTGACAGTGAGCGCGCAGCTAATAT | 8. |
| | | TCTCAAGTATTAGTGAAGCCACAGATGTAA | |
| | | TACTTGAGAATATTAGCTGCTTGCCTACTG | |
| | | CCTCGGA | |
| | V3LHS_324769 | TGCTGTTGACAGTGAGCGACAGCCTGTTCG | 9. |
| | | GCGAGGACAATAGTGAAGCCACAGATGTAT | |
| | | TGTCCTCGCCGAACAGGCTGCTGCCTACTG | |
| | | CCTCGGA | |
| CASP8 | TRCN0000003575 | CCGGGAATCACAGACTTTGGACAAACTCGA | 10. |
| | | GTTTGTCCAAAGTCTGTGATTCTTTTT | |
| | TRCN0000003579 | CCGGGCCTTGATGTTATTCCAGAGACTCGA | 11. |
| | | GTCTCTGGAATAACATCAAGGCTTTTT | |
| CLK2 | TRCN0000000752 | CCGGGAAAGCATAAGCGACGAAGAACTCGA | 12. |
| | | GTTCTTCGTCGCTTATGCTTTCTTTTT | |
| | TRCN0000010543 | CCGGGTGGAGTATAGGCTGCATCATCTCGA | 13. |
| | | GATGATGCAGCCTATACTCCACTTTTT | |
| DEPDC7 | TRCN0000135313 | CCGGCCTAACCAAGACAGTCAGTTACTCGA | 14. |
| | | GTAACTGACTGTCTTGGTTAGGTTTTTTG | |
| | TRCN0000134293 | CCGGCTACTGTATTTCATGGCTGTTCTCGA | 15. |
| | | GAACAGCCATGAAATACAGTAGTTTTTTG | |
| EBF1 | TRCN0000013828 | CCGGGCTCTATACAAGGGACACTATCTCGA | 16. |
| | | GATAGTGTCCCTTGTATAGAGCTTTTT | |
| | TRCN0000013829 | CCGGCCCTCAGATCCAGTGATAATTCTCGA | 17. |
| | | GAATTATCACTGGATCTGAGGGTTTTT | |
| GEMIN4 | TRCN0000007894 | CCGGGCTCTCCCAGTTTAGTGCAATCTCGA | 18. |
| | | GATTGCACTAAACTGGGAGAGCTTTTT | |
| | TRCN0000007895 | CCGGGTTTGTTTACACCCAGGTGTTCTCGA | 19. |
| | | GAACACCTGGGTGTAAACAAACTTTTT | |
| KHDC4 | TRCN0000121825 | CCGGGAGCTAAACAACAGATGCCATCTCGA | 20. |
| (aka | | GATGGCATCTGTTGTTTAGCTCTTTTTTG | |
| BLOM7) | TRCN0000142264 | CCGGGCTATACACAACCCTCTGCTACTCGA | 21. |
| | | GTAGCAGAGGGTTGTGTATAGCTTTTTTG | |
| IFI16 | TRCN0000019080 | CCGGGACAGGACAATGTCACAATATCTCGA | 22. |
| | | GATATTGTGACATTGTCCTGTCTTTTT | |
| | TRCN0000019082 | CCGGCCAAAGAAGATCATTGCCATACTCGA | 23. |
| | | GTATGGCAATGATCTTCTTTGGTTTTT | |
| MMP8 | TRCN0000052096 | CCGGGCAACCAGTATCAGTCTACAACTCGA | 24. |
| | | GTTGTAGACTGATACTGGTTGCTTTTTG | |
| | TRCN0000052097 | CCGGCCAAGATATTACGCATTTGATCTCGA | 25. |
| | | GATCAAATGCGTAATATCTTGGTTTTTG | |
| NKIRAS1 | TRCN0000047524 | CCGGGTGAATAACCTTGAATCCTTTCTCGA | 26. |
| | | GAAAGGATTCAAGGTTATTCACTTTTTG | |
| | TRCN0000047527 | CCGGCTCTGATTGAACCATTCACTTCTCGA | 27. |
| | | GAAGTGAATGGTTCAATCAGAGTTTTTG | |
| PAGE1 | TRCN0000115709 | CCGGCTGACGAAGTGGAATCACCAACTCGA | 28. |
| | | GTTGGTGATTCCACTTCGTCAGTTTTTG | |
| | TRCN0000115711 | CCGGCAGGATTCTACACCTGCTGAACTCGA | 29. |
| | | GTTCAGCAGGTGTAGAATCCTGTTTTTG | |
| PARP14 | TRCN0000053162 | CCGGGCACCATTTGAAGAGTCACTACTCGA | 30. |
| | | GTAGTGACTCTTCAAATGGTGCTTTTTG | |
| | TRCN0000053158 | CCGGCGGAACTTCATTCTTCACAAACTCGA | 31. |
| | | GTTTGTGAAGAATGAAGTTCCGTTTTTG | |
| POLR2K | TRCN0000021879 | CCGGGTGGAGAGTGTCACACAGAAACTCGA | 32. |
| | | GTTTCTGTGTGACACTCTCCACTTTTT | |
| | TRCN0000021880 | CCGGCAGAGAATGTGGATACAGAATCTCGA | 33. |
| | | GATTCTGTATCCACATTCTCTGTTTTT | |
| PRKCG | TRCN0000002326 | CCGGCCGATATTCTCCCTGACCTTACTCGA | 34. |
| | | GTAAGGTCAGGGAGAATATCGGTTTTT | |
| | TRCN0000002325 | CCGGGTGGAATGAGACCTTTGTGTTCTCGA | 35. |
| | | GAACACAAAGGTCTCATTCCACTTTTT | |
| PYM1 | TRCN0000136900 | CCGGCTTGAGCAGGACTCTTGATAACTCGA | 36. |
| (aka | | GTTATCAAGAGTCCTGCTCAAGTTTTTTG | |
| WIBG) | TRCN0000138190 | CCGGCCAAACGTAACCTGAAGCGAACTCGA | 37. |
| | | GTTCGCTTCAGGTTACGTTTGGTTTTTTG | |
| RACK1 | TRCN0000006472 | CCGGGATGTGGTTATCTCCTCAGATCTCGA | 38. |
| (aka | | GATCTGAGGAGATAACCACATCTTTTT | |
| GNB2L1) | V2LHS_69420 | TGCTGTTGACAGTGAGCGAAGCATCAAGAT | 39. |
| | | CTGGGATTTATAGTGAAGCCACAGATGTAT | |
| | | AAATCCCAGATCTTGATGCTGTGCCTACTG | |
| | | CCTCGGA | |
| RRAGD | TRCN0000059533 | CCGGCGGCAAGTCGTCTATTCAGAACTCGA | 40. |
| | | GTTCTGAATAGACGACTTGCCGTTTTTG | |
| | TRCN0000059537 | CCGGCCAGGGCCTACAAAGTGAATACTCGA | 41. |
| | | GTATTCACTTTGTAGGCCCTGGTTTTTG | |
| RRP12 | TRCN0000157888 | CCGGCGACTATGTTCCCAGTGAGAACTCGA | 42. |
| | | GTTCTCACTGGGAACATAGTCGTTTTTTG | |
| | TRCN0000156901 | CCGGGATGACTTGGAACTAGGGCTTCTCGA | 43. |
| | | GAAGCCCTAGTTCCAAGTCATCTTTTTTG | |
| SENP7 | TRCN0000004544 | CCGGGCAGTGATTGTGGAGTATATTCTCGA | 44. |
| | | GAATATACTCCACAATCACTGCTTTTT | |
| | TRCN0000004545 | CCGGGTCGAATATGTCAGTACCAAACTCGA | 45. |
| | | GTTTGGTACTGACATATTCGACTTTTT | |
| SOS1 | TRCN0000048145 | CCGGGCACTTTATTTGCAGTCAATACTCGA | 46. |
| | | GTATTGACTGCAAATAAAGTGCTTTTTG | |
| | TRCN0000048143 | CCGGCCCTAGAAATAGAACCACGAACTCGA | 47. |
| | | GTTCGTGGTTCTATTTCTAGGGTTTTTG | |
| STYK1 | TRCN0000001745 | CCGGCAGGGACACAAAGGGAGAAATCTCGA | 48. |
| | | GATTTCTCCCTTTGTGTCCCTGTTTTT | |
| | TRCN0000001746 | CCGGCGCCTAGAAGCTGCCATTAAACTCGA | 49. |
| | | GTTTAATGGCAGCTTCTAGGCGTTTTT | |
| TICAM1 | TRCN0000123201 | CCGGCCTACTTCTCACCTCCAACTTCTCGA | 50. |
| | | GAAGTTGGAGGTGAGAAGTAGGTTTTTG | |
| | TRCN0000123203 | CCGGTCCCTGGAATCATCATCGGAACTCGA | 51. |
| | | GTTCCGATGATGATTCCAGGGATTTTTG | |
| TNIK | TRCN0000037514 | CCGGCGGTAGAAGAAGGTCAAAGATCTCGA | 52. |
| | | GATCTTTGACCTTCTTCTACCGTTTTTG | |
| | TRCN0000037515 | CCGGCCAGAAGTTATTGCCTGTGATCTCGA | 53. |
| | | GATCACAGGCAATAACTTCTGGTTTTTG | |
| TGFB2 | TRCN0000033427 | CCGGCCAAGATTGAACAGCTTTCTACTCGA | 54. |
| | | GTAGAAAGCTGTTCAATCTTGGTTTTTG | |
| | TRCN0000033428 | CCGGCACACTCGATATGGACCAGTTCTCGA | 55. |
| | | GAACTGGTCCATATCGAGTGTGTTTTTG | |
| YES1 | TRCN0000001608 | CCGGGCAGTTAATTTCAGCAGTCTTCTCGA | 56. |
| | | GAAGACTGCTGAAATTAACTGCTTTTT | |
| | TRCN0000001609 | CCGGCTGCACTGTATGGTCGGTTTACTCGA | 57. |
| | | GTAAACCGACCATACAGTGCAGTTTTT | |
| ZNF74 | TRCN0000017546 | CCGGCCTCCACTGCACAAGCCAGATCTCGA | 58. |
| | | GATCTGGCTTGTGCAGTGGAGGTTTTT | |
| | TRCN0000017547 | CCGGACACCTGCTCAGCACATACTACTCGA | 59. |
| | | GTAGTATGTGCTGAGCAGGTGTTTTTT | |

To determine whether the KRAS-EFs promote KRAS expression at the transcriptional or post-transcriptional level, we monitored KRAS expression by quantitative RT-PCR (qRT-PCR). FIG. 3C shows that knockdown of the majority of KRAS-EFs did not reduce KRAS mRNA levels, where knockdown of two KRAS-EFs, AR and PARP14, substantially reduced KRAS mRNA levels, suggesting their function was transcriptional.

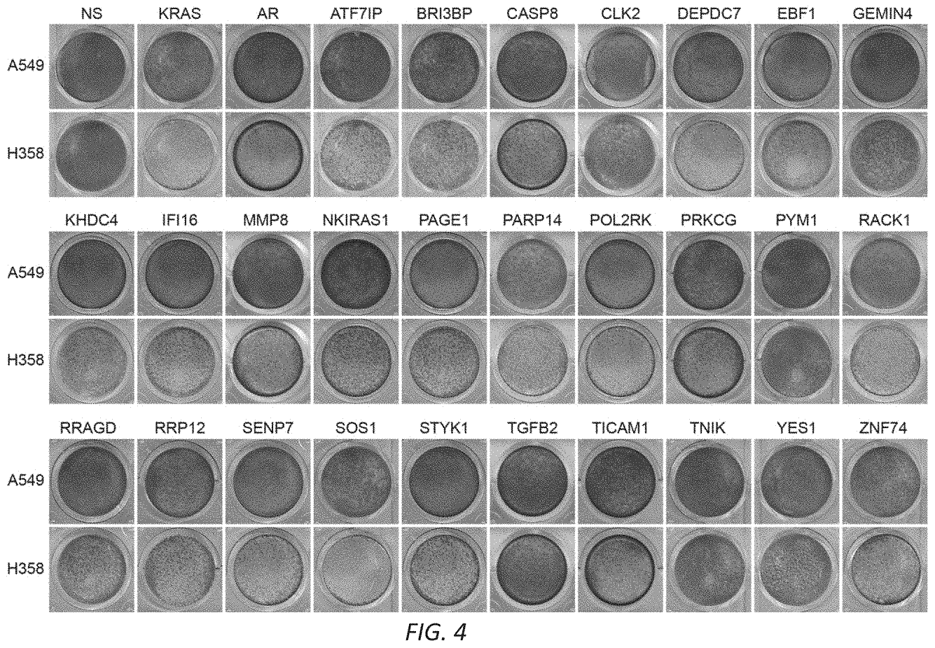

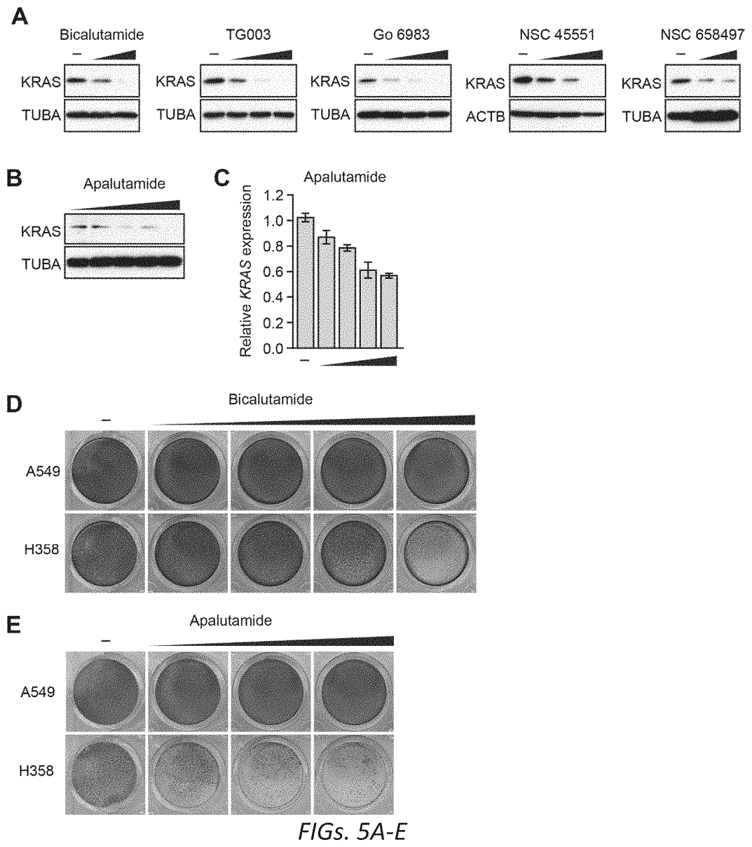

We found, as expected, that shRNA-mediated knockdown of each of the KRAS-EFs reduced proliferation of KRAS-dependent H358 human lung cancer cells (harboring a heterozygous KRAS(G12C) mutation) but not KRAS-independent A549 cells (FIG. 4).

Several of the KRAS-EFs are druggable and small molecule inhibitors are available (Table A). We therefore sought to determine whether small molecule inhibitors could, like shRNA-mediated knockdown, reduce KRAS expression. We obtained inhibitors against several of the KRAS-EFs: AR (bicalutamide; Cayman Chemical), CLK2 (TG003; Cayman Chemical), PKCγ (Go 6983; Cayman Chemical), SENP7 (NSC 45551; NCI/DTP Open Chemical Repository), and SOS1 (NSC 658497; NCI/DTP Open Chemical Repository). FIG. 5A shows that treatment of A549 cells with any one of these small molecule inhibitors reduced KRAS protein levels in a dose-dependent manner.

One KRAS-EF we found of particular interest is AR, a transcription factor that is activated by binding of androgens. In brief, androgen binding induces a conformational change in the AR resulting in dissociation of associated heat shock proteins, dimerization, phosphorylation and translocation to the nucleus (Gao et al. 2007, Chem Rev 105:3352-3370). AR signaling is required for the normal development and maintenance of the prostate. Notably, aberrant AR signaling drives the growth of nearly all prostate cancers. Accordingly, a number of AR small molecule antagonists have been developed and approved for the treatment of prostate cancer including bicalutamide, flutamide, nilutamide, enzalutamide, darolutamide and apalutamide (Helsen et al. 2014, Endocr Relat Cancer 21:T105-18). Notably, apalutamide (also called Erleada) is a second-generation AR antagonist that was approved by the FDA in February 2018 for the treatment of non-metastatic castration-resistant prostate cancer (fda.gov/drugs/informationondrugs/approveddrugs/ucm596796.htm). In addition to prostate cancers, in which AR is a known driver of tumor growth, AR has also been found to be expressed in a number of solid tumors including lung, colorectal and pancreas (Munoz et al. 2015, Oncotarget 6:592-603; Schweizer and Yu 2017, Cancers (Basel) 9:7). Recently, this conclusion has been confirmed by RNA-seq results of solid tumors, which is one component of the analysis performed by The Cancer Genome Atlas (TCGA; cancer.gov/about-nci/organization/ccg/research/structural-genomics/tcga).

We found that AR antagonist apalutamide reduced KRAS protein (FIG. 5B) and mRNA (FIG. 5C) levels in a dose-dependent manner in A549 cells. Furthermore, biculatamide (FIG. 5D) and apalutamide (FIG. 5E) reduced proliferation of KRAS-dependent H358 cells but not KRAS-independent A549 cells.

We asked whether AR was functioning directly by binding to and stimulating transcription of KRAS. We performed bioinformatic analysis, which revealed the presence of several candidate AR-binding sites in the KRAS promoter and within intron 1 conforming to either an androgen response element (ARE) half-site (5′-AGAACA-3′; Denayer et al. 2010, Mol Endocrinol 24:898-913) or a 6-basepair AR-binding site motif (5′-AGAACC-3′; Massie et al. 2007, EMBO Rep 8:871-878) (FIG. 6A). We then performed chromatin immunoprecipitation (ChIP) experiments and found that AR binds to the KRAS promoter and at multiple sites within intron 1 in A549 cells (FIG. 6B). As expected, treatment with the AR antagonist apalutamide, which sequesters AR in the cytoplasm (Rice, Malhotra and Stoyanova 2019, Front Oncol 8:801), substantially reduced AR binding to all sites within KRAS. To confirm that binding of AR stimulated KRAS expression, we performed CRISPR-based genome editing in A549 cells to construct deletion mutants in each of the AR-binding sites in KRAS. Using this strategy, we generated homozygous AR-binding site deletion mutants in Intronl-1 (Intron1-1Δ) and Intron1-2 (Intron1-2Δ); for Intron1-3 we generated a homozygous mutant deleting the second AR-binding site (Intron1-3Δ) (FIG. 6C). FIG. 6D shows that each of these AR-binding site deletion mutants has significantly reduced expression of KRAS compared to that of the wild-type KRAS gene. Thus, binding of AR to each of these sites stimulates KRAS expression.

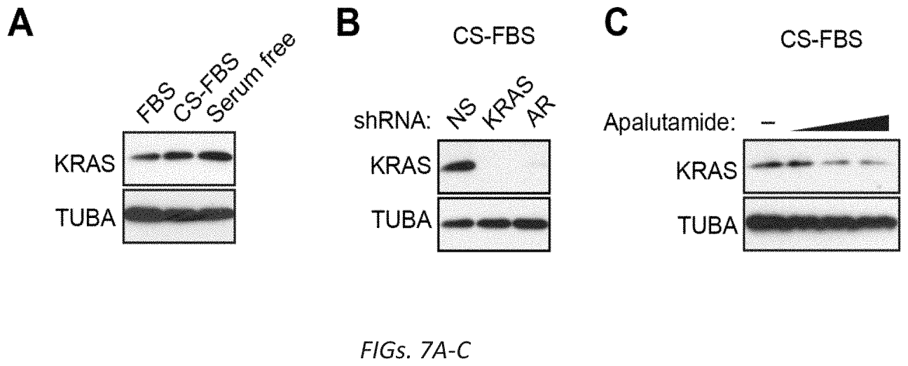

In addition to canonical activation by androgens, AR can also function through several ligand-independent mechanisms, which typically involves activation of AR by phosphorylation (Weigel and Zhang 1998, J Mol Med 76:469-479). The experiments described above were carried out without addition of exogenous androgens, suggesting that AR activation was ligand independent. However, it remained possible that ligand-dependent activation occurred due to androgen present in the serum used to culture cells. To address this possibility we cultured A549 cells in fetal bovine serum (FBS), charcoal stripped-FBS (CS-FBS), which removes androgens (Cao et al. 2009, Endocr Res 34:101-8; Krycer and Brown 2013, PLoS One 8:e54007), or under serum-free conditions, and monitored KRAS expression by immunoblotting. We reasoned that if AR promoted KRAS expression in a ligand-dependent manner then KRAS expression should be reduced in medium lacking androgen. Alternatively, if AR promoted KRAS expression in a ligand-independent manner then KRAS expression should not change in the absence of androgen. The results of FIG. 7A show that KRAS expression was not decreased when A549 cells were cultured in CS-FBS or under serum free conditions compared to that obtained in cells cultured in FBS. Moreover, shRNA-mediated knockdown (FIG. 7B) or pharmacological inhibition (FIG. 7C) of AR in A549 cells cultured in CS-FBS resulted in decreased KRAS levels, indicating that AR stimulated KRAS expression in the absence of androgen. Collectively, these results indicate that KRAS expression is regulated by AR through a ligand-independent signaling pathway in A549 cells.

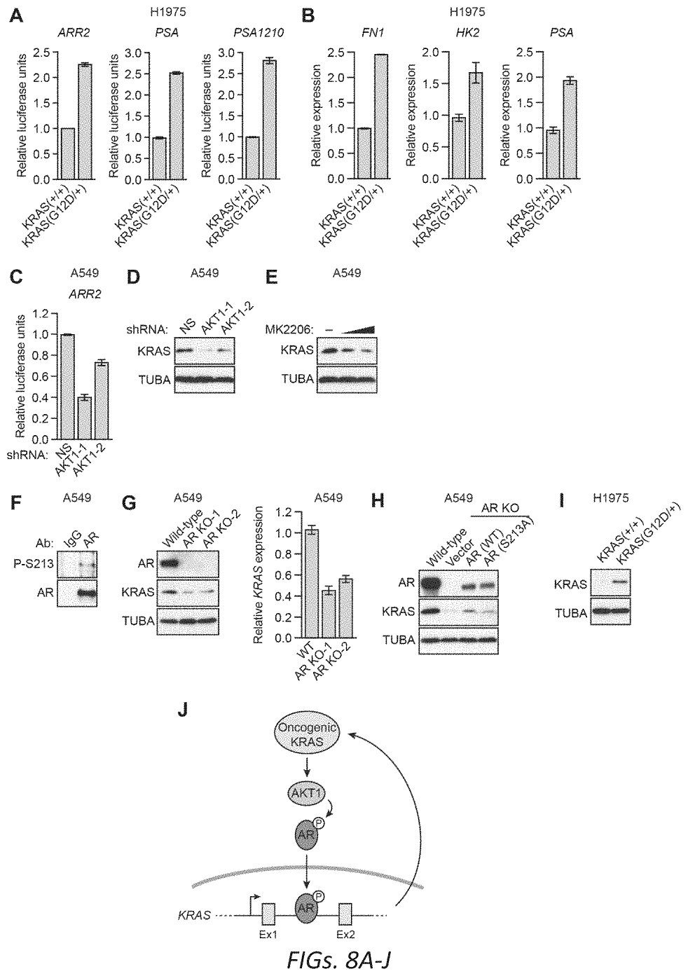

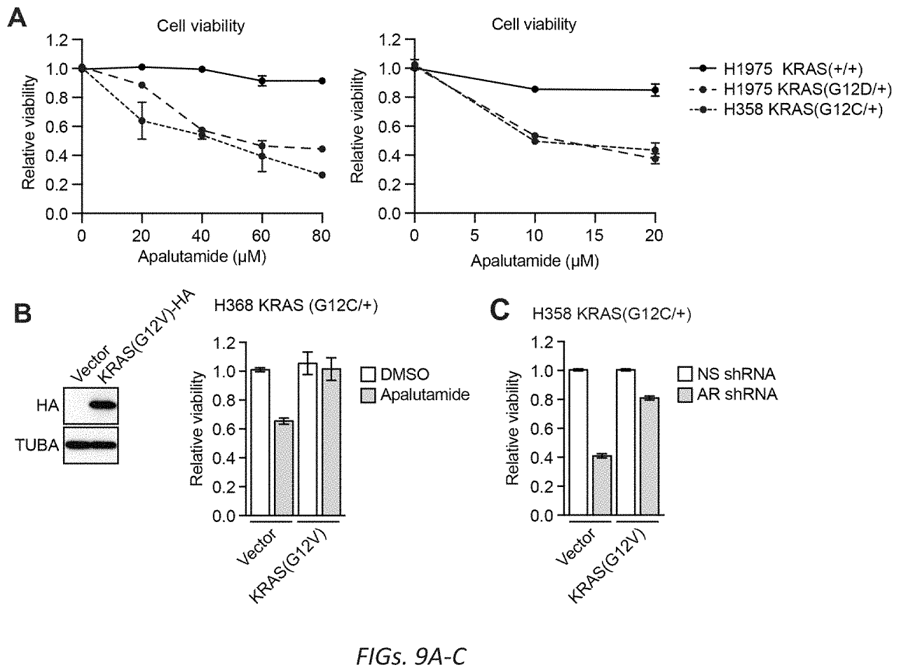

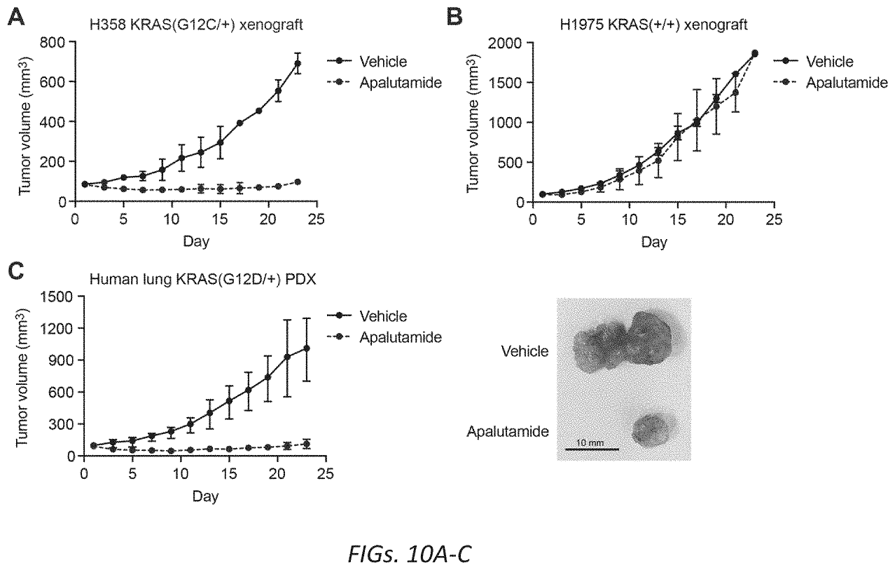

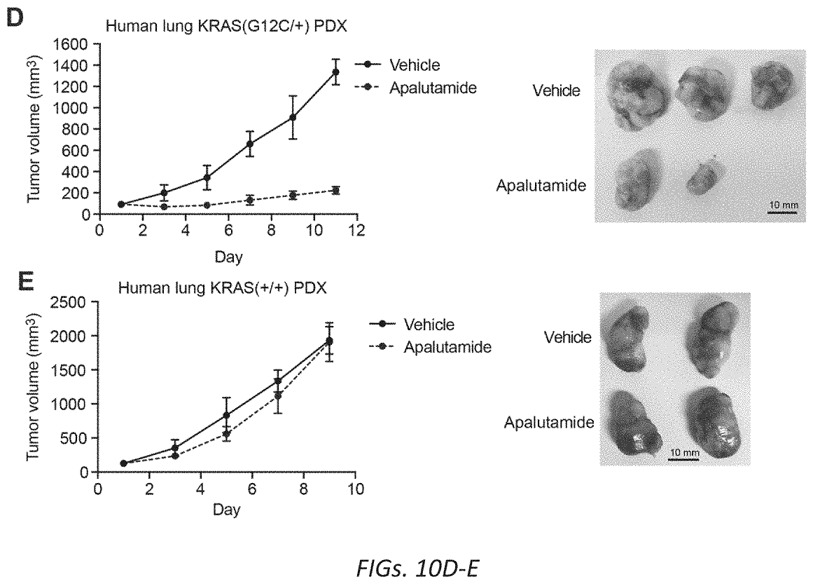

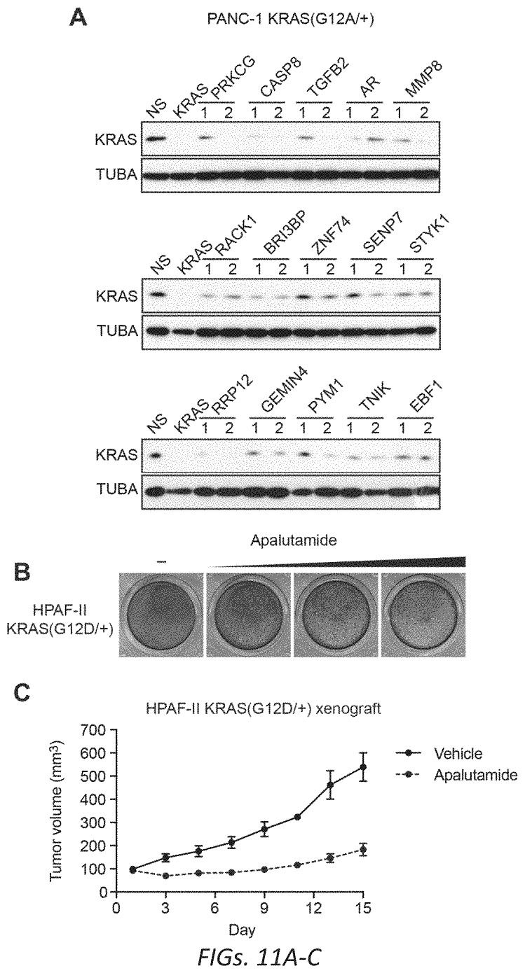

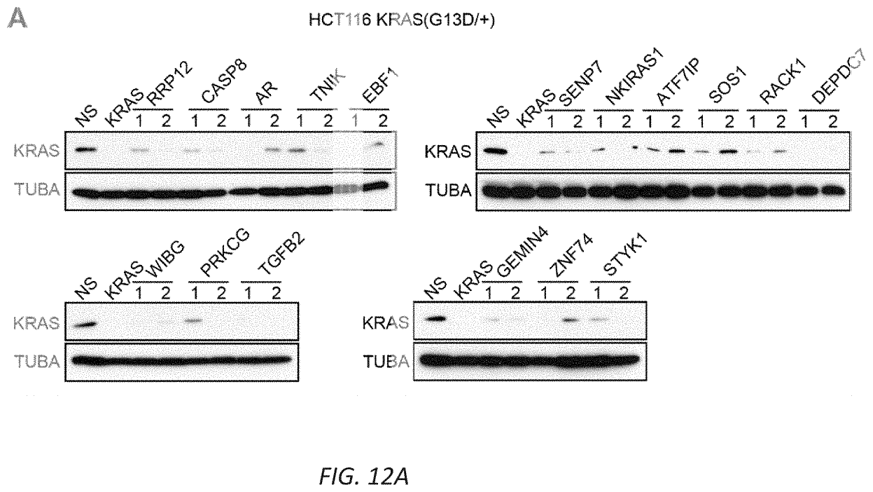

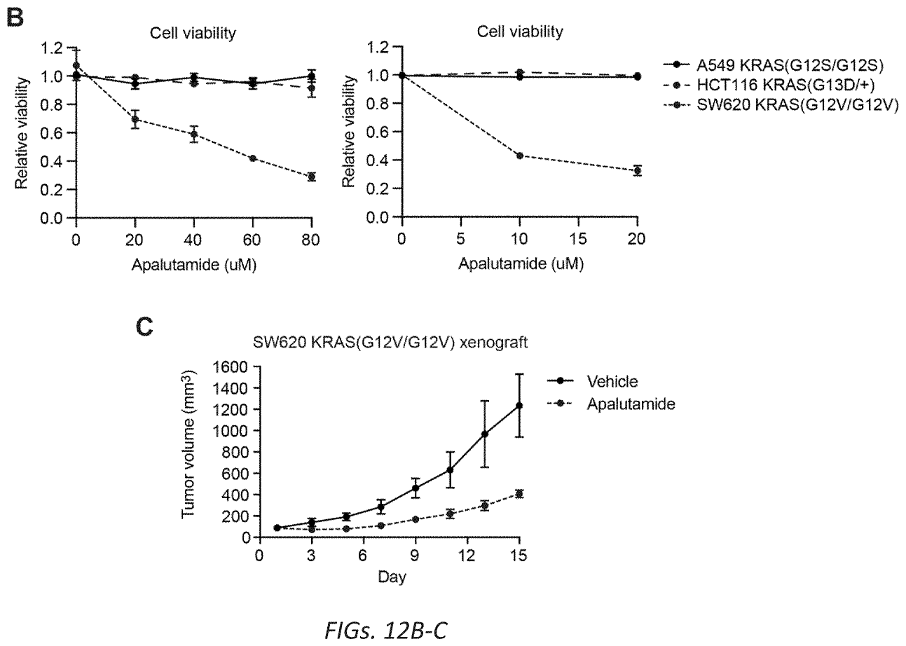

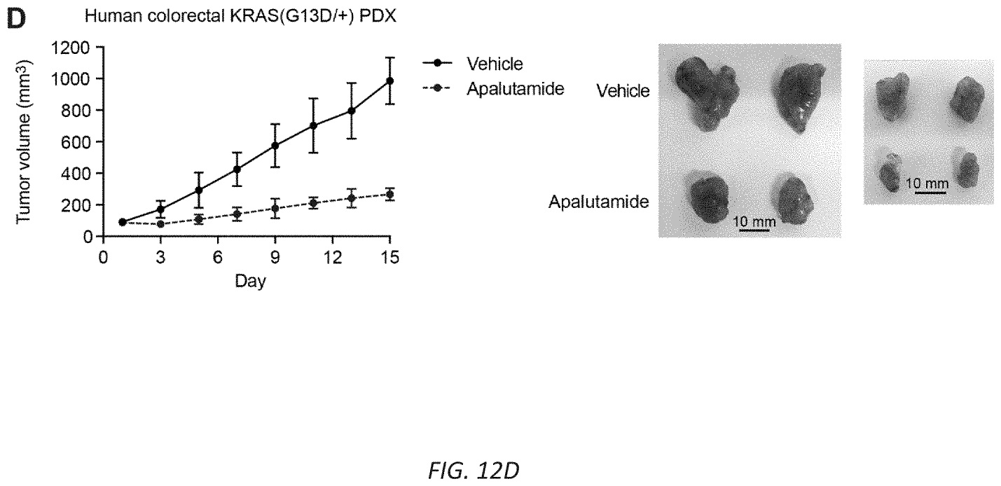

The results described above led us to hypothesize that the ligand-independent activation of AR was promoted by oncogenic KRAS. As a first test of this idea, we analyzed AR activity in an isogenic H1975 human non-small cell lung cancer cell line pair that contain either wild-type KRAS (H1975 KRAS(+/+)), or a heterozygous KRAS(G12D) allele (H1975 KRAS(G12D/+)). We found that transfected AR reporter genes (FIG. 8A) had a higher level of expression in H1975 KRAS(G12D/+) cells compared to H1975 KRAS(+/+) cells, indicative of increased AR activity. Likewise, expression of representative endogenous AR target genes was higher in H1975 KRAS(G12D/+) cells compared to H1975 KRAS(+/+) cells (FIG. 8B).

As mentioned above, phosphorylation of AR by a protein kinase is a common mechanism of ligand-independent AR activation. AKT1, a serine-threonine protein kinase that is component of a proliferative signaling pathway that functions downstream of KRAS, has been shown to phosphorylate and activate AR (Wen et al. 2000, Cancer Res 60:6841-5). ShRNA-mediated knockdown of AKT1 in A549 cells reduced expression of an AR reporter gene (FIG. 8C), indicative of decreased AR activity, and also resulted in reduced KRAS levels (FIG. 8D). Reduced KRAS levels were also observed following treatment of A549 cells with the AKT1 inhibitor MK2206 (Yan 2009, Cancer Res 69:9) (FIG. 8E).

AKT1 is known to phosphorylate AR at S213 (Wen et al. 2000, Cancer Res 60:6841-5). We first confirmed that AR is phosphorylated at S213 in A549 cells (FIG. 8F). To elucidate the role of AR S213 phosphorylation in expression of KRAS we first constructed a homozygous AR deletion mutant in A549 cells using CRISPR/Cas9 genome editing. FIG. 8G shows, as expected, that A549 AR knockout (KO) cells expressed lower levels of KRAS than parental A549 cells. We then transfected A549 AR KO cells with a vector expressing either wild-type AR or an AR(S213A) mutant, which cannot undergo phosphorylation at S213. FIG. 8H shows that transfection of wild-type AR resulted in higher levels of KRAS than the AR(S213A) mutant, demonstrating a role for S213 phosphorylation in AR-mediated stimulation of KRAS expression.

The collective results described above suggested that in cells containing oncogenic KRAS AR is activated and binds to and stimulates transcription of the KRAS gene. A prediction of this idea is that KRAS expression levels will be higher in cells containing oncogenic KRAS than in cells containing wild-type KRAS. In support of this prediction, FIG. 8I shows that KRAS levels are higher in H1975 KRAS(G12D/+) cells than in H1975 KRAS(+/+) cells. Also in support of this prediction, a study analyzing expression profiling data from The Cancer Genome Atlas (TCGA) found that tumors containing oncogenic KRAS expressed higher levels of KRAS than comparable tumors containing wild-type KRAS {Stephens, Yi, Kessing, Niossley and McCormick 2017, Cancer Inform 16:1176935117711944). FIG. 8I shows our current model for AR-mediated promotion of oncogenic KRAS expression.

We next analyzed the ability of the AR antagonist apalutamide to kill oncogenic KRAS-dependent human lung cancer cell lines and suppress growth of oncogenic KRAS-dependent tumors. We found that apalutamide, in a dose-dependent manner, reduced viability of human lung cancer cell lines containing oncogenic KRAS (H358 KRAS(G12C/+) and H1975 KRAS(G12D/+)) but not those containing wild-type KRAS (H1975 KRAS(+/+)) (FIG. 9A). To confirm that the loss of viability was due to decreased KRAS levels we asked whether ectopic expression of oncogenic KRAS would counteract the effects of apalutamide treatment. Toward that end we derived an H358 KRAS(G12C/+) cell line stably expressing oncogenic KRAS(G12V) or as a control containing only the empty expression vector (FIG. 9B. left). FIG. 9B (right) shows that loss of viability that normally occurs following apalutamide treatment was not observed in H358 KRAS(G12C/+) cells ectopically expressing KRAS(G12V). Likewise, ectopic expression of KRAS(G12V) counteracted the loss of viability following shRNA-mediated knockdown of AR in H358 KRAS(G12C/+) cells (FIG. 9C). These results indicate that the loss of viability following AR inhibition is entirely, or in large part, due to decreased levels of oncogenic KRAS.

We next determined the ability of AR antagonists to suppress tumor growth. These experiments were performed in female mice to avoid the potentially confounding effects of endogenous androgen levels in young male mice. In the first set of experiments, H358 KRAS(G12C/+) cells were injected subcutaneously into the flanks of female nude mice, and when tumors reached ˜100 mm³ mice were treated with either vehicle or apalutamide, which were administered by oral gavage. FIG. 10A shows that apalutamide markedly suppressed tumor growth of H358 KRAS(G12C/+) xenografts. By contrast, apalutamide had no effect on tumor growth on H1975 KRAS(+/+) xenografts (FIG. 10B).

In a second set of experiments we asked whether AR antagonists would suppress growth of tumors originating from patient derived xenografts (PDXs) containing oncogenic KRAS. We obtained two lung PDXs, one harboring KRAS(G12D/+) and another harboring KRAS(G12C/+), and transplanted them subcutaneously into 5 mice. As above, when subcutaneous PDX-derived tumors reached ˜100 mm³, mice were treated with vehicle or apalutamide. The results of FIGS. 10C and 10D show that treatment with apalutamide arrested growth of tumors derived from a human lung PDX containing an oncogenic KRAS(G12D) or KRAS(G12C) mutation. By contrast, apalutamide did not significantly affect tumor growth of a KRAS(+/+) human lung PDX (FIG. 10E).

Finally, to determine the generality of our results to other oncogenic KRAS-positive tumor types, we performed a subset of the key experiments with human colorectal and pancreatic cancer cells. We found that knockdown of the KRAS-EFs reduced KRAS protein levels in PANC-1 KRAS(G12A/+) human pancreatic cancer cells (FIG. 11A). Apalutamide treatment reduced viability of a KRAS-dependent human pancreatic cancer cell line, HPAF-II (KRAS(G12D/+) (FIG. 11B) and suppressed tumor growth of HPAF-II (KRAS(G12D/+) xenografts (FIG. 11C). Similarly, knockdown of KRAS-EFs reduced KRAS protein levels in HCT116 KRAS(G13D/+) human colorectal cancer cells (FIG. 12A). Apalutamide treatment reduced viability of a KRAS-dependent human colorectal cancer cell line, SW620 KRAS(G12V/G12V) (FIG. 12B) and suppressed tumor growth of SW620 KRAS(G12V/G12V) xenografts (FIG. 12C).

EXEMPLARY SEQUENCES
KRAS Promoter (comprises 1 kb upstream of the
transcription start site)
Bold and double underline indicates the CCTTCT
sequence shown in FIG. 6A
(SEQ ID NO: 60)
TTATCAACACAGACTCCGGGTATGCTAGCATGTTTAATTGCCCCATTGTT
TAATGTCTTAACTCCACGAACTTTAACTGATTAATCTGTCTTCTAATTAA
TGTTTGAATGACTCTCCTCAGGTCTAAACTACCAAGGCCATCTCTACTTA
AAAACAGTTGTCTTTTGTTTGTGATTTCAGGGGCCCTGGGTATAAGCGAA
GTCCCTGTTTAGAGACCTTGTGATGGGTTCAAAATATCAAGAAAGATAGC
AAAATATCACAAGCCTCCTGACCCGAGAAGATTAGCGTTGAAAGGGTCTG
TCGTGTTTGTTTGGGCCTGGGGCTAAATTCCCAGCCCAAGTGCTGAGGCT
GATAATAATCGGGGCGGCGATCAGACAGCCCCGGTGTGGGAAATCGTCCG
CCCGGTCTCCCTAAGTCCCCGAAGTCGCCTCCCACTTTTGGTGACTGCTT
GTTTATTTACATGCAGTCAATGATAGTAAATGGATGCGCGCCAGTATAGG
CCGACCCTGAGGGTGGCGGGGTGCTCTTCGCAGCTTCTCTGTGGAGACCG
GTCAGCGGGGCGGCGTGGCCGCTCGCGGCGTCTCCCTGGTGGCATCCGCA
CAGCCCGCCGCGGTCCGGTCCCGCTCCGGGTCAGAATTGGCGGCTGCGGG
GACAGCCTTGCGGCTAGGCAGGGGGCGGGCCGCCGCGTGGGTCCGGCAGT
CCCTCCTCCCGCCAAGGCGCCGCCCAGACCCGCTCTCCAGCCGGCCCGGC
TCGCCACCCTAGACCGCCCCAGCCACCCCTTCCTCCGCCGGCCCGGCCCC
CGCTCCTCCCCCGCCGGCCCGGCCCGGCCCCCT [symbol] CCCCGCCGG
CGCTCGCTGCCTCCCCCTCTTCCCTCTTCCCACACCGCCCTCAGCCGCTC
CCTCTCGTACGCCCGTCTGAAGAAGAATCGAGCGCGGAACGCATCGATAG
CTCTGCCCTCTGCGGCCGCCCGGCCCCGAACTCATCGGTGTGCTCGGAGC
TCGATTT
KRAS Intron 1
Bold indicates the CCTTCT sequence shown in
FIG. 6A
Bold and double underline indicates the half-site
AGAACA sequence shown in FIG. 6A
(SEQ ID NO: 61)
GTACGGAGCGGACCACCCCTCCTGGGCCCCTGCCCGGGTCCCGACCCTCT
TTGCCGGCGCCGGGCGGGGCCGGCGGCGAGTGAATGAATTAGGGGTCCCC
GGAGGGGCGGGTGGGGGGCGCGGGCGCGGGGTCGGGGCGGGCTGGGTGAG
AGGGGTCTGCAGGGGGGAGGCGCGCGGACGCGGCGGCGCGGGGAGTGAGG
AATGGGCGGTGCGGGGCTGAGGAGGGTGAGGCTGGAGGCGGTCGCCGCTG
GTGCTGCTTCCTGGACGGGGAACCCCTTCCTTCCTCCTCCCCGAGAGCCG
CGGCTGGAGGCTTCTGGGGAGAAACTCGGGCCGGGCCGGCTGCCCCTCGG
AGCGGTGGGGTGCGGTGGAGGTTACTCCCGCGGCGCCCCGGCCTCCCCTC
CCCCTCTCCCCGCTCCCGCACCTCTTGCCTCCCTTTCCAGCACTCGGCTG
CCTCGGTCCAGCCTTCCCTGCTGCATTTGGCATCTCTAGGACGAAGGTAT
AAACTTCTCCCTCGAGCGCAGGCTGGACGGATAGTGGTCCTTTTCCGTGT
GTAGGGGATGTGTGAGTAAGAGGGGAGGTCACGTTTTGGAAGAGCATAGG
AAAGTGCTTAGAGACCACTGTTTGAGGTTATTGTGTTTGGAAAAAAATGC
ATCTGCCTCCGAGTTCCTGAATGCTCCCCTCCCCCATGTATGGGCTGTGA
CATTGCTGTGGCCACAAAGGAGGAGGTGGAGGTAGAGATGGTGGA [symbol]
GGTGGCCAACACCCTACACGTAGAGCCTGTGACCTACAGTGAA
AAGGAAAAAGTTAATCCCAGATGGTCTGTTTTGCTTGGTCAAGTTAAACC
CGAAGAAAACCCGCAGAGCAGAAGCAAGGCTTTTTCCTTGCTAGTTGAGT
GTAGACAGCAATAGCAAAAATAGTACTTGAAGTTTAATTTACCTGTTCTT
GTCCTTTCCCCTATTTCTTATGTATTACCCTCATCCCCTCGTCTCTTTTA
TACTACCCTCATTTTGCAGATGTGTTCTACATCTCAAGAGTTATTACAGT
ACTCCAAAACAGCACTTACATGATTTTTTAAACTTACAGAGGAATTGTAG
CAATCCACCAGCTAACCGCCTGAAATAGACTTAAACATGTGCATCTCCTT
TTTTTTTTTTTTTTTGAGACACAGTCTCGCTCTGTTGCCCAGGCTGGAGT
GCAATGGCGCGGTATCGGCTCACTGAAACCTCCGCCTCCTGGGTTCAAGC
AATTCTCCTGCCTCAGCCTCCCGAGTAGCTGGGACTAGTAGGTGCACGCC
ACCATGCCCAGCTAATTTTTGTATTTTTAGTAGAGACAGAGTTTCATCAT
GTTGGTCAGGATGGTCTCCATCTGCTCTGTTGCCCAGGCTGGAGTGCAGT
GGCGCCGTCTCGGCTCACTGCAACCTCTGCCTCCTGCATTCAAGCAATTC
TCCTGCCTCAGCCTCCCGAATAACTGGGATTACAGGTGTCTGCTGCCATG
CCCGGCTAATTTTTTGTATTTTTAGTAGAGACGGGGGTTTCACCATGTTG
GTCAGGCTGGTCTAGAACTCCTGACCTCGTGATCTGCCCGCCTCGGCCTC
CCACAGTGGCATGTGCATCTTATAGCTGAAGTCTAAGCCTTCTTAAATCT
TGAGATCCATCAAAACAGACAGGTTTTCTAATTGTTATACAATGTATATG
TTATGTTTATAATAGAAATCATTTTACAAATAAGTTATAAATGGGAAAGG
TCTATTTGTAATTATCAGCTCAGAATTAACCATAAAACTGGTGTCACTGA
AGTGACTGAGGTCCAAAATGCTGACTCTGCATGTTATAGACTACAGATAT
CAAATATGGTTGCTAACAATAGTTTACTTTGAGACTGTAGCCATCCACAG
TATATTTGCTTTTAAGAGATGGTAGATGGTAATTCAGTTTTATGAAAAAT
AAAAATGAATTTTCTTCCATTACAAAATTGTTGGATTCGAGTCCAGTCCA
CTCCTTACTAGCTTTTCTAACTCTCGGTGAGGGATCCCCTCCCAGCCCAT
GATCTTCATTTGGTAAGACTCCTTTGGAACCCAGTTCTCTCTAGTGGATT
TAAATGTGATTTGGTTTTAAAAATCTCATTCAAGGAATTTTTTTTTTTTC
TGGAAACAACCACCGCATAAACAAGTAAACCGGAAGATACATGTGGCTCT
GAATTCATATATATACACAAACTCTAATCCAATGTCTGTCCACAGTATTT
CCTAGGCTAGTAAACTTTTTGGCCTTAACGACCCCTCTACCCTCTTTGTT
TTTTTGAGAGAGAGAGTCTCACTCTGTCACCCAGGCCGGAATGCAGTGGC
GCGATCTCGGCCCGCTACTACCTCCGACTCTCAGGCTCAAGCGATTCTCC
CGCCTCAGCTTCCCGAGTAGCCGGGATTACAGGCTCCCGCCACCGGGCTA
ATTGTATTTTTAGATACGGGATTTCACCATGTTGGCCAGGCTGGTCTCGA
CCTCCTGACCTCAGGTGATCCGCCCGCCTAAGCCTCCCAAAGTGCTGGGA
TTACAGGCCACCACACCCGGCCTACACTCTTAAAAATTATCGAAGGGGCC
GGGCACATTGGCTCTTATCTGTAATCCCAGCACTTTGGGAGACTGAGGCG
GGAGGATCGCTTGAGGCCAGGAGTTGGAGACCAGCGTACTCAACATAGTG
AGACCTTGTTATAAAGAAAAAAAAAATCCAGGATTAAAAAAAATCTTTGA
TTTGTTTGGGATTTATTAATATTTACCGTATTGGAAATTAAAACAATTTT
TTAAAATGTATTCATTTAAAAATAATAAGCCCATTACTTGGTAACATGAA
TAAAATATTTTATGAAAAATAACTATTTTCCAAAACAAAACCAAAACTTA
GAAAAGTGGTATTGTTTCACACTTCAGTAAATCTCTTTAATGATGTGGCT
TAATAGAAGATATGGATTCTTATATCTGCATCTGCATTCAATCTATTATG
ATCACACATCTGGAAAACTTGTGAAAGAATGGGAGTTAAAAGGGTAAAGG
ACATCTTAATGTTATTATGAAAACAGTTTTGACCTCTTGCACACCAGAAA
AGTCTTAGTAACCTGAGGGGTTCCTAGACCACATTTTGAGAACTGTTTTA
GGCTATGCAAACTGGTTGGGGGGAGGTTGGGGTAGGCAGAGAGCTAGAAG
ATACATTTTAGTGTAATTCTCCTCATCTATTCCTAATTGCTTTGGCCTAC
ATTTGAAATAAAGCGTGGAGGCAAACGGGATAAGATACATGTTTGTAGTG
GTTGTTAACTTCACCCTAGACAAGCAGCCAATAAGTCTAGGTAGAGCAGA
GTAAGGCGGGGAACTATGCCGTGACCGTGTGTGATACAATTTTTCTAGCC
TGTGGTGCTTTTTGCGGCAGGGCTTAGGAGTAAGGTTAGTATGTTATCAT
TTGGGAAACCAAATTATTATTTTGGGTCTTCAGTCAATTATGATGCTGTG
TATATTTAGTGTTTATCTACAATATATGCACATTCATTAATTTGGAGCTA
CTCATCCTATAATAAATAGTTGTGCATTTACTCCCATTTTTTTCTGCATT
TCTCTCCTTATTTATAATTATGTGTTACATGAGGGAAAGGAGGTGAAATT
AAACATTCATATTATTTCAAAAAATTTGAAACAACTAACTAAAAAATATG
TTTTATTTTCTGTATGGTGTTTGTTATACAATCTGTCAATATTCATGCAC
CTCTTGGGAGACAGTGTATGAAAAGCAAAGAGTAACAGTCACATGGATTA
CTGATTACTGAGATATATTCACTTGCATCTTTTTTTTTTTTTGAGACGGA
GTGGCTCTGTCGCCCAGGCTGGAGTGCAGTGGCGTGATCTCGGCTCACTG
CAAGCTCCGCCTCCTGGGTTCACGCCATTCTTCTGCCTCAGCCTCCCAAG
TAGCTGGGACTACAGGCGCCCGCCACCACGCCCGGCTAATTTTTTTATAT
TTTTAGTAGAGACGGGGTTTCACCGGGTTAGCCAGGATGGTCTTGATCTC
CTGACCTCGTGATCCACCCTCCTCGGCCTCCCAAAGTGCTAGGATTATAG
GCGTGAGCCACCGTGCCCGGCTCACTTGCATCTCTTAACAGCTGTTTTCT
TACTAAAAACAGTGTTTATCTCTAATCTTTTTGTTTGTTTGTTTGTTTTG
AGATGGAGTCTTACTCCGTCACCCAATCTGGAGTGCAGTGGCGTGATCTG
GGCTCACTGCAACCTCTGCCTCCCGGGTTCAAGTGATTCTCCTTCCTCAG
CCTCCCCAGTAGCTAGGACTACAGGAGAGCGCCACCACGCCTGATTAATT
TTTGTATTTTTAGTAGAGAGAGGGTTTCACCATATTGGCCAGGCTGGTCT
TGAACTCCTGGCCTCAGGTGATCCACCCGCCTTGGCCTCTGAAAGTGCTG
GGATTACAGGCATGAGCCGCCGCACCCGGCTTTCTAATCTTTATCTTTTT
TTGTGCAGCGGTGATACAGGATTATGTATTGTACTGAACAGTTAATTCGG
AGTTCTCTTGGTTTTTAGCTTTATTTTCCCCAGAGATTTTTTTTTTTTTT
TTTTTTTTTGAGACGGAGTCTTGCTCTATCGCCAGGCTGGAGTGCAGTGG
CGCCATCTCGGCTCATTGCAACCTCGGACTCCTATTTTCCCCAGAGATAT
TTCACACATTAAAATGTCGTCAAATATTGTTCTTCTTTGCCTCAGTGTTT
AAATTTTTATTTCCCCATGACACAATCCAGCTTTATTTGACACTCATTCT
CTCAACTCTCATCTGATTCTTACTGTTAATATTTATCCAAGAGAACTACT
GCCATGATGCTTTAAAAGTTTTTCTGTAGCTGTTGCATATTGACTTCTAA
CACTTAGAGGTGGGGGTCCACTAGGAAAACTGTAACAATAAGAGTGGAGA
TAGCTGTCAGCAACTTTTGTGAGGGTGTGCTACAGGGTGTAGAGCACTGT
GAAGTCTCTACATGAGTGAAGTCATGATATGATCCTTTGAGAGCCTTTAG
CCGCCGCAGAACAGCAGTCTGGCTATTTAGATAGAACAACTTGATTTTAA
GATAAAAGAACTGTCTATGTAGCATTTATGCATTTTTCTTAAGCGTCGAT
GGAGGAGTTTGTAAATGAAGTACAGTTCATTACGATACACGTCTGCAGTC
AACTGGAATTTTCATGATTGAATTTTGTAAGGTATTTTGAAATAATTTTT
CATATAAAGGTGAGTTTGTATTAAAAGGTACTGGTGGAGTATTTGATAGT
GTATTAACCTTATGTGTGACATGTTCTAATATAGTCACATTTTCATTATT
TTTATTATAAG

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has a cancer containing an oncogenic KRAS mutant, the method comprising administering to the subject a therapeutically effective amount of an inhibitor of a KRAS-EF listed in Table 1, optionally in combination with a therapeutically effective amount of one or more chemotherapeutic and/or immunotherapeutic agents.

2. The method of claim 1, wherein the inhibitor is a small molecule antagonist of Androgen Receptor (AR).

3. The method of claim 2, wherein the small molecule antagonist of AR is a non-steroidal antagonist, optionally a diarylthiohydantoin derivative, preferably apalutamide, proxalutamide, enzalutamide, RD-162, flutamide, nilutamide, bicalutamide, topilutamide, AZD3514, darolutamide, or a diarylhydantoin; a steroidal androgen receptor antagonist, optionally a 17α-Hydroxyprogesterone derivative, a 19-norprogesterone derivative, 19-Nortestosterone derivative, or a 17α-Spirolactone derivative; a progestin that has direct androgen receptor antagonistic activity, optionally medrogestone, promegestone, or trimegestone; an N-Terminal domain antiandrogen, optoinally bisphenol A, EPI-001, ralaniten, or JN compound; EZN-4176, AZD-5312, apatorsen, galeterone, ODM-2014, TRC-253, or BMS-641988.

4. The method of claim 1, wherein the inhibitor is a small molecule inhibitor of a KRAS-EF, optionally a small molecule inhibitor listed in Table A.

5. The method of claim 1, wherein the inhibitor is an inhibitory nucleic acid targeting a KRAS-EF, optionally an inhibitory nucleic acid listed in Table B.

6. The method of claim 5, wherein the inhibitory nucleic acid is an antisense oligonucleotide, siRNA, or shRNA.

7. The method of claim 5, wherein the inhibitory nucleic acid targets AR.

8. The method of claim 1, wherein the inhibitory nucleic acid targets inhibits binding of AR to the KRAS promoter and/or first intron.

9. The method of claim 8, wherein the inhibitory nucleic acid is a triplex forming oligo (TFO) that binds to the KRAS promoter and/or first intron.

10. The method of claim 8, wherein the inhibitory nucleic acid comprises a decoy sequence that binds to AR.

11. The method of claim 1, wherein the inhibitor is a targeted protein degrader comprising a first ligand that binds to a KRAS-EF and a second ligand that binds to a E3 ubiquitin ligase, with a linker therebetween.

12. The method of claim 11, wherein the targeted protein degrader is ARV-110, ARD-69, ARD-61, or ARCC-4.

13. The method of claim 1, further comprising identifying the subject as having a cancer containing an oncogenic KRAS mutant.

14. The method of claim 13, wherein identifying the subject comprises determining the presence of a mutation in KRAS in the cancer.

15. The method of claim 14, wherein determining the presence of a mutation comprises:
obtaining a sample comprising a cell from the cancer; and
detecting the presence of a mutation associated with cancer in a KRAS gene in the cell.

16. The method of claim 15, wherein the mutation is G12, G13, and/or Q61.

17.-27. (canceled)

28. A method of identifying a candidate compound for the treatment of a cancer containing an oncogenic KRAS mutant, the method comprising:
providing a sample comprising a nucleic acid comprising a sequence comprising a promotor plus intron 1 of the KRAS gene, preferably promotor plus intron 1 of an oncogenic KRAS gene, and AR protein;
contacting the sample with a test compound;
measuring binding of the AR protein to the nucleic acid in the presence and absence of the test compound; and
selecting a test compound that decreases binding of the AR to the nucleic acid as a candidate compound for the treatment of cancer containing an oncogenic KRAS mutant.

29. The method of claim 28, wherein the sample is a cell expressing a reporter construct comprising a fusion of a promotor plus intron 1 of an oncogenic KRAS gene and a detectable protein, optionally a fluorescent protein.


SEQUENCE LISTING (61 sequences)

SEQ ID NO: 1 - DNA, 17 bp, Artificial Sequence
  SELEX-derived ARE oligo
agaacatctcgtgtacc

SEQ ID NO: 2 - DNA, 15 bp, Artificial Sequence
  inverted repeat (IR)-ARE oligo
agaacagcaagtact

SEQ ID NO: 3 - DNA, 15 bp, Artificial Sequence
  in vivo-derived direct repeat (DR)-ARE oligo
agaactggaagagct

SEQ ID NO: 4 - DNA, 57 bp, Artificial Sequence
  Oligo ID TRCN0000003715
ccggcctgctaatcaagtcacacatctcgagatgtgtgacttgattagcaggttttt

SEQ ID NO: 5 - DNA, 57 bp, Artificial Sequence
  Oligo ID TRCN0000003717
ccggcgcgactactacaactttccactcgagtggaaagttgtagtagtcgcgttttt

SEQ ID NO: 6 - DNA, 57 bp, Artificial Sequence
  Oligo ID TRCN0000020828
ccggcgtcgatatatggaagaagaactcgagttcttcttccatatatcgacgttttt

SEQ ID NO: 7 - DNA, 57 bp, Artificial Sequence
  Oligo ID TRCN0000020825
ccggccagggactttggtgactaatctcgagattagtcaccaaagtccctggttttt

SEQ ID NO: 8 - DNA, 97 bp, Artificial Sequence
  Oligo ID V2LHS_71753
tgctgttgacagtgagcgcgcagctaatattctcaagtattagtgaagccacagatgtaatacttgagaatattagctgcttgcctactgcctcgga

SEQ ID NO: 9 - DNA, 97 bp, Artificial Sequence
  Oligo ID V3LHS_324769
tgctgttgacagtgagcgacagcctgttcggcgaggacaatagtgaagccacagatgtattgtcctcgccgaacaggctgctgcctactgcctcgga

SEQ ID NO: 10 - DNA, 57 bp, Artificial Sequence
  Oligo ID TRCN0000003575
ccgggaatcacagactttggacaaactcgagtttgtccaaagtctgtgattcttttt

SEQ ID NO: 11 - DNA, 57 bp, Artificial Sequence
  Oligo ID TRCN0000003579
ccgggccttgatgttattccagagactcgagtctctggaataacatcaaggcttttt

SEQ ID NO: 12 - DNA, 57 bp, Artificial Sequence
  Oligo ID TRCN0000000752
ccgggaaagcataagcgacgaagaactcgagttcttcgtcgcttatgctttcttttt

SEQ ID NO: 13 - DNA, 57 bp, Artificial Sequence
  Oligo ID TRCN0000010543
ccgggtggagtataggctgcatcatctcgagatgatgcagcctatactccacttttt

SEQ ID NO: 14 - DNA, 59 bp, Artificial Sequence
  Oligo ID TRCN0000135313
ccggcctaaccaagacagtcagttactcgagtaactgactgtcttggttaggttttttg

SEQ ID NO: 15 - DNA, 59 bp, Artificial Sequence
  Oligo ID TRCN0000134293
ccggctactgtatttcatggctgttctcgagaacagccatgaaatacagtagttttttg

SEQ ID NO: 16 - DNA, 57 bp, Artificial Sequence
  Oligo ID TRCN0000013828
ccgggctctatacaagggacactatctcgagatagtgtcccttgtatagagcttttt

SEQ ID NO: 17 - DNA, 57 bp, Artificial Sequence
  Oligo ID TRCN0000013829
ccggccctcagatccagtgataattctcgagaattatcactggatctgagggttttt

SEQ ID NO: 18 - DNA, 57 bp, Artificial Sequence
  Oligo ID TRCN0000007894
ccgggctctcccagtttagtgcaatctcgagattgcactaaactgggagagcttttt

SEQ ID NO: 19 - DNA, 57 bp, Artificial Sequence
  Oligo ID RCN0000007895
ccgggtttgtttacacccaggtgttctcgagaacacctgggtgtaaacaaacttttt

SEQ ID NO: 20 - DNA, 59 bp, Artificial Sequence
  Oligo ID TRCN0000121825
ccgggagctaaacaacagatgccatctcgagatggcatctgttgtttagctcttttttg

SEQ ID NO: 21 - DNA, 59 bp, Artificial Sequence
  Oligo ID TRCN0000142264
ccgggctatacacaaccctctgctactcgagtagcagagggttgtgtatagcttttttg

SEQ ID NO: 22 - DNA, 57 bp, Artificial Sequence
  Oligo ID TRCN0000019080
ccgggacaggacaatgtcacaatatctcgagatattgtgacattgtcctgtcttttt

SEQ ID NO: 23 - DNA, 57 bp, Artificial Sequence
  Oligo ID TRCN0000019082
ccggccaaagaagatcattgccatactcgagtatggcaatgatcttctttggttttt

SEQ ID NO: 24 - DNA, 58 bp, Artificial Sequence
  Oligo ID TRCN0000052096
ccgggcaaccagtatcagtctacaactcgagttgtagactgatactggttgctttttg

SEQ ID NO: 25 - DNA, 58 bp, Artificial Sequence
  Oligo ID TRCN0000052097
ccggccaagatattacgcatttgatctcgagatcaaatgcgtaatatcttggtttttg

SEQ ID NO: 26 - DNA, 58 bp, Artificial Sequence
  Oligo ID TRCN0000047524
ccgggtgaataaccttgaatcctttctcgagaaaggattcaaggttattcactttttg

SEQ ID NO: 27 - DNA, 58 bp, Artificial Sequence
  Oligo ID TRCN0000047527
ccggctctgattgaaccattcacttctcgagaagtgaatggttcaatcagagtttttg

SEQ ID NO: 28 - DNA, 58 bp, Artificial Sequence
  Oligo ID TRCN0000115709
ccggctgacgaagtggaatcaccaactcgagttggtgattccacttcgtcagtttttg

SEQ ID NO: 29 - DNA, 58 bp, Artificial Sequence
  Oligo ID TRCN0000115711
ccggcaggattctacacctgctgaactcgagttcagcaggtgtagaatcctgtttttg

SEQ ID NO: 30 - DNA, 58 bp, Artificial Sequence
  Oligo ID TRCN0000053162
ccgggcaccatttgaagagtcactactcgagtagtgactcttcaaatggtgctttttg

SEQ ID NO: 31 - DNA, 58 bp, Artificial Sequence
  Oligo ID TRCN0000053158
ccggcggaacttcattcttcacaaactcgagtttgtgaagaatgaagttccgtttttg

SEQ ID NO: 32 - DNA, 57 bp, Artificial Sequence
  Oligo ID TRCN0000021879
ccgggtggagagtgtcacacagaaactcgagtttctgtgtgacactctccacttttt

SEQ ID NO: 33 - DNA, 57 bp, Artificial Sequence
  Oligo ID TRCN0000021880
ccggcagagaatgtggatacagaatctcgagattctgtatccacattctctgttttt

SEQ ID NO: 34 - DNA, 57 bp, Artificial Sequence
  Oligo ID TRCN0000002326
ccggccgatattctccctgaccttactcgagtaaggtcagggagaatatcggttttt

SEQ ID NO: 35 - DNA, 57 bp, Artificial Sequence
  Oligo ID TRCN0000002325
ccgggtggaatgagacctttgtgttctcgagaacacaaaggtctcattccacttttt

SEQ ID NO: 36 - DNA, 59 bp, Artificial Sequence
  Oligo ID TRCN0000136900
ccggcttgagcaggactcttgataactcgagttatcaagagtcctgctcaagttttttg

SEQ ID NO: 37 - DNA, 59 bp, Artificial Sequence
  Oligo ID TRCN0000138190
ccggccaaacgtaacctgaagcgaactcgagttcgcttcaggttacgtttggttttttg

SEQ ID NO: 38 - DNA, 57 bp, Artificial Sequence
  Oligo ID TRCN0000006472
ccgggatgtggttatctcctcagatctcgagatctgaggagataaccacatcttttt

SEQ ID NO: 39 - DNA, 97 bp, Artificial Sequence
  Oligo ID V2LHS_69420
tgctgttgacagtgagcgaagcatcaagatctgggatttatagtgaagccacagatgtataaatcccagatcttgatgctgtgcctactgcctcgga

SEQ ID NO: 40 - DNA, 58 bp, Artificial Sequence
  Oligo ID TRCN0000059533
ccggcggcaagtcgtctattcagaactcgagttctgaatagacgacttgccgtttttg

SEQ ID NO: 41 - DNA, 58 bp, Artificial Sequence
  Oligo ID TRCN0000059537
ccggccagggcctacaaagtgaatactcgagtattcactttgtaggccctggtttttg

SEQ ID NO: 42 - DNA, 59 bp, Artificial Sequence
  Oligo ID TRCN0000157888
ccggcgactatgttcccagtgagaactcgagttctcactgggaacatagtcgttttttg

SEQ ID NO: 43 - DNA, 59 bp, Artificial Sequence
  Oligo ID TRCN0000156901
ccgggatgacttggaactagggcttctcgagaagccctagttccaagtcatcttttttg

SEQ ID NO: 44 - DNA, 57 bp, Artificial Sequence
  Oligo ID TRCN0000004544
ccgggcagtgattgtggagtatattctcgagaatatactccacaatcactgcttttt

SEQ ID NO: 45 - DNA, 57 bp, Artificial Sequence
  Oligo ID TRCN0000004545
ccgggtcgaatatgtcagtaccaaactcgagtttggtactgacatattcgacttttt

SEQ ID NO: 46 - DNA, 58 bp, Artificial Sequence
  Oligo ID TRCN0000048145
ccgggcactttatttgcagtcaatactcgagtattgactgcaaataaagtgctttttg

SEQ ID NO: 47 - DNA, 58 bp, Artificial Sequence
  Oligo ID TRCN0000048143
ccggccctagaaatagaaccacgaactcgagttcgtggttctatttctagggtttttg

SEQ ID NO: 48 - DNA, 57 bp, Artificial Sequence
  Oligo ID TRCN0000001745
ccggcagggacacaaagggagaaatctcgagatttctccctttgtgtccctgttttt

SEQ ID NO: 49 - DNA, 57 bp, Artificial Sequence
  Oligo ID TRCN0000001746
ccggcgcctagaagctgccattaaactcgagtttaatggcagcttctaggcgttttt

SEQ ID NO: 50 - DNA, 58 bp, Artificial Sequence
  Oligo ID TRCN0000123201
ccggcctacttctcacctccaacttctcgagaagttggaggtgagaagtaggtttttg

SEQ ID NO: 51 - DNA, 58 bp, Artificial Sequence
  Oligo ID TRCN0000123203
ccggtccctggaatcatcatcggaactcgagttccgatgatgattccagggatttttg

SEQ ID NO: 52 - DNA, 58 bp, Artificial Sequence
  Oligo ID TRCN0000037514
ccggcggtagaagaaggtcaaagatctcgagatctttgaccttcttctaccgtttttg

SEQ ID NO: 53 - DNA, 58 bp, Artificial Sequence
  Oligo ID TRCN0000037515
ccggccagaagttattgcctgtgatctcgagatcacaggcaataacttctggtttttg

SEQ ID NO: 54 - DNA, 58 bp, Artificial Sequence
  Oligo ID TRCN0000033427
ccggccaagattgaacagctttctactcgagtagaaagctgttcaatcttggtttttg

SEQ ID NO: 55 - DNA, 58 bp, Artificial Sequence
  Oligo ID TRCN0000033428
ccggcacactcgatatggaccagttctcgagaactggtccatatcgagtgtgtttttg

SEQ ID NO: 56 - DNA, 57 bp, Artificial Sequence
  Oligo ID TRCN0000001608
ccgggcagttaatttcagcagtcttctcgagaagactgctgaaattaactgcttttt

SEQ ID NO: 57 - DNA, 57 bp, Artificial Sequence
  Oligo ID TRCN0000001609
ccggctgcactgtatggtcggtttactcgagtaaaccgaccatacagtgcagttttt

SEQ ID NO: 58 - DNA, 57 bp, Artificial Sequence
  Oligo ID TRCN0000017546
ccggcctccactgcacaagccagatctcgagatctggcttgtgcagtggaggttttt

SEQ ID NO: 59 - DNA, 57 bp, Artificial Sequence
  Oligo ID TRCN0000017547
ccggacacctgctcagcacatactactcgagtagtatgtgctgagcaggtgtttttt

SEQ ID NO: 60 - DNA, 1005 bp, Artificial Sequence
  KRAS Promoter
ttatcaacacagactccgggtatgctagcatgtttaattgccccattgtttaatgtcttaactccacgaactttaactgattaatctgtcttctaattaatgtttgaatgactctcctcaggtctaaactaccaaggccatctctacttaaaaacagttgtcttttgtttgtgatttcaggggccctgggtataagcgaagtccctgtttagagaccttgtgatgggttcaaaatatcaagaaagatagcaaaatatcacaagcctcctgacccgagaagattagcgttgaaagggtctgtcgtgtttgtttgggcctggggctaaattcccagcccaagtgctgaggctgataataatcggggcggcgatcagacagccccggtgtgggaaatcgtccgcccggtctccctaagtccccgaagtcgcctcccacttttggtgactgcttgtttatttacatgcagtcaatgatagtaaatggatgcgcgccagtataggccgaccctgagggtggcggggtgctcttcgcagcttctctgtggagaccggtcagcggggcggcgtggccgctcgcggcgtctccctggtggcatccgcacagcccgccgcggtccggtcccgctccgggtcagaattggcggctgcggggacagccttgcggctaggcagggggcgggccgccgcgtgggtccggcagtccctcctcccgccaaggcgccgcccagacccgctctccagccggcccggctcgccaccctagaccgccccagccaccccttcctccgccggcccggcccccgctcctcccccgccggcccggcccggccccctccttctccccgccggcgctcgctgcctccccctcttccctcttcccacaccgccctcagccgctccctctcgtacgcccgtctgaagaagaatcgagcgcggaacgcatcgatagctctgccctctgcggccgcccggccccgaactcatcggtgtgctcggagctcgattt

SEQ ID NO: 61 - DNA, 5355 bp, Artificial Sequence
  KRAS Intron 1
gtacggagcggaccacccctcctgggcccctgcccgggtcccgaccctctttgccggcgccgggcggggccggcggcgagtgaatgaattaggggtccccggaggggcgggtggggggcgcgggcgcggggtcggggcgggctgggtgagaggggtctgcaggggggaggcgcgcggacgcggcggcgcggggagtgaggaatgggcggtgcggggctgaggagggtgaggctggaggcggtcgccgctggtgctgcttcctggacggggaaccccttccttcctcctccccgagagccgcggctggaggcttctggggagaaactcgggccgggccggctgcccctcggagcggtggggtgcggtggaggttactcccgcggcgccccggcctcccctccccctctccccgctcccgcacctcttgcctccctttccagcactcggctgcctcggtccagccttccctgctgcatttggcatctctaggacgaaggtataaacttctccctcgagcgcaggctggacggatagtggtccttttccgtgtgtaggggatgtgtgagtaagaggggaggtcacgttttggaagagcataggaaagtgcttagagaccactgtttgaggttattgtgtttggaaaaaaatgcatctgcctccgagttcctgaatgctcccctcccccatgtatgggctgtgacattgctgtggccacaaaggaggaggtggaggtagagatggtggaagaacaggtggccaacaccctacacgtagagcctgtgacctacagtgaaaaggaaaaagttaatcccagatggtctgttttgcttggtcaagttaaacccgaagaaaacccgcagagcagaagcaaggctttttccttgctagttgagtgtagacagcaatagcaaaaatagtacttgaagtttaatttacctgttcttgtcctttcccctatttcttatgtattaccctcatcccctcgtctcttttatactaccctcattttgcagatgtgttctacatctcaagagttattacagtactccaaaacagcacttacatgattttttaaacttacagaggaattgtagcaatccaccagctaaccgcctgaaatagacttaaacatgtgcatctcctttttttttttttttttgagacacagtctcgctctgttgcccaggctggagtgcaatggcgcggtatcggctcactgaaacctccgcctcctgggttcaagcaattctcctgcctcagcctcccgagtagctgggactagtaggtgcacgccaccatgcccagctaatttttgtatttttagtagagacagagtttcatcatgttggtcaggatggtctccatctgctctgttgcccaggctggagtgcagtggcgccgtctcggctcactgcaacctctgcctcctgcattcaagcaattctcctgcctcagcctcccgaataactgggattacaggtgtctgctgccatgcccggctaattttttgtatttttagtagagacgggggtttcaccatgttggtcaggctggtctagaactcctgacctcgtgatctgcccgcctcggcctcccacagtggcatgtgcatcttatagctgaagtctaagccttcttaaatcttgagatccatcaaaacagacaggttttctaattgttatacaatgtatatgttatgtttataatagaaatcattttacaaataagttataaatgggaaaggtctatttgtaattatcagctcagaattaaccataaaactggtgtcactgaagtgactgaggtccaaaatgctgactctgcatgttatagactacagatatcaaatatggttgctaacaatagtttactttgagactgtagccatccacagtatatttgcttttaagagatggtagatggtaattcagttttatgaaaaataaaaatgaattttcttccattacaaaattgttggattcgagtccagtccactccttactagcttttctaactctcggtgagggatcccctcccagcccatgatcttcatttggtaagactcctttggaacccagttctctctagtggatttaaatgtgatttggttttaaaaatctcattcaaggaattttttttttttctggaaacaaccaccgcataaacaagtaaaccggaagatacatgtggctctgaattcatatatatacacaaactctaatccaatgtctgtccacagtatttcctaggctagtaaactttttggccttaacgacccctctaccctctttgtttttttgagagagagagtctcactctgtcacccaggccggaatgcagtggcgcgatctcggcccgctactacctccgactctcaggctcaagcgattctcccgcctcagcttcccgagtagccgggattacaggctcccgccaccgggctaattgtatttttagatacgggatttcaccatgttggccaggctggtctcgacctcctgacctcaggtgatccgcccgcctaagcctcccaaagtgctgggattacaggccaccacacccggcctacactcttaaaaattatcgaaggggccgggcacattggctcttatctgtaatcccagcactttgggagactgaggcgggaggatcgcttgaggccaggagttggagaccagcgtactcaacatagtgagaccttgttataaagaaaaaaaaaatccaggattaaaaaaaatctttgatttgtttgggatttattaatatttaccgtattggaaattaaaacaattttttaaaatgtattcatttaaaaataataagcccattacttggtaacatgaataaaatattttatgaaaaataactattttccaaaacaaaaccaaaacttagaaaagtggtattgtttcacacttcagtaaatctctttaatgatgtggcttaatagaagatatggattcttatatctgcatctgcattcaatctattatgatcacacatctggaaaacttgtgaaagaatgggagttaaaagggtaaaggacatcttaatgttattatgaaaacagttttgacctcttgcacaccagaaaagtcttagtaacctgaggggttcctagaccacattttgagaactgttttaggctatgcaaactggttggggggaggttggggtaggcagagagctagaagatacattttagtgtaattctcctcatctattcctaattgctttggcctacatttgaaataaagcgtggaggcaaacgggataagatacatgtttgtagtggttgttaacttcaccctagacaagcagccaataagtctaggtagagcagagtaaggcggggaactatgccgtgaccgtgtgtgatacaatttttctagcctgtggtgctttttgcggcagggcttaggagtaaggttagtatgttatcatttgggaaaccaaattattattttgggtcttcagtcaattatgatgctgtgtatatttagtgtttatctacaatatatgcacattcattaatttggagctactcatcctataataaatagttgtgcatttactcccatttttttctgcatttctctccttatttataattatgtgttacatgagggaaaggaggtgaaattaaacattcatattatttcaaaaaatttgaaacaactaactaaaaaatatgttttattttctgtatggtgtttgttatacaatctgtcaatattcatgcacctcttgggagacagtgtatgaaaagcaaagagtaacagtcacatggattactgattactgagatatattcacttgcatctttttttttttttgagacggagtggctctgtcgcccaggctggagtgcagtggcgtgatctcggctcactgcaagctccgcctcctgggttcacgccattcttctgcctcagcctcccaagtagctgggactacaggcgcccgccaccacgcccggctaatttttttatatttttagtagagacggggtttcaccgggttagccaggatggtcttgatctcctgacctcgtgatccaccctcctcggcctcccaaagtgctaggattataggcgtgagccaccgtgcccggctcacttgcatctcttaacagctgttttcttactaaaaacagtgtttatctctaatctttttgtttgtttgtttgttttgagatggagtcttactccgtcacccaatctggagtgcagtggcgtgatctgggctcactgcaacctctgcctcccgggttcaagtgattctccttcctcagcctccccagtagctaggactacaggagagcgccaccacgcctgattaatttttgtatttttagtagagagagggtttcaccatattggccaggctggtcttgaactcctggcctcaggtgatccacccgccttggcctctgaaagtgctgggattacaggcatgagccgccgcacccggctttctaatctttatctttttttgtgcagcggtgatacaggattatgtattgtactgaacagttaattcggagttctcttggtttttagctttattttccccagagatttttttttttttttttttttttgagacggagtcttgctctatcgccaggctggagtgcagtggcgccatctcggctcattgcaacctcggactcctattttccccagagatatttcacacattaaaatgtcgtcaaatattgttcttctttgcctcagtgtttaaatttttatttccccatgacacaatccagctttatttgacactcattctctcaactctcatctgattcttactgttaatatttatccaagagaactactgccatgatgctttaaaagtttttctgtagctgttgcatattgacttctaacacttagaggtgggggtccactaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataag